(19) United States
(12) Patent Application Publication
(10) Pub. No.: US 2025/0006366 A1
(43) Pub. Date: Jan. 2, 2025

(54) ARTIFICIAL INTELLIGENCE-AIDED CLASSIFICATION SYSTEM FOR ALZHEIMER'S DISEASE SCREENING FROM RETINAL PHOTOGRAPHS

(71) Applicant: The Chinese University of Hong Kong, Hong Kong (CN)

(72) Inventors: Yim Lui Carol CHEUNG, Hong Kong (CN); Anran RAN, Hong Kong (CN); Shujun WANG, Hong Kong (CN); Chung Tong Vincent MOK, Hong Kong (CN); Chee Yung Clement THAM, Hong Kong (CN)

(21) Appl. No.: 18/344,190

(22) Filed: Jun. 29, 2023

(51) Int. Cl. G16H 50/20

(52) U.S. Cl. CPC G16H 50/20

(57) ABSTRACT

The subject invention pertains to an artificial intelligence-aided classification system for Alzheimer's disease (AD) screening from retinal photographs, which includes a three-in-one deep learning (DL)-based pre-diagnosis module for image assessment and a DL-based AD classification module with additional heatmaps for visualization. For the AD classification module, three kinds of DL models predict the AD-dementia/non-demented probabilities from three directions with both eyes' four images (Direction-1), both eyes' four images combing demographical information (Direction-2), and a single eye's two images (Direction-3), respectively. For the pre-diagnosis image assessment, embodiments provide one pre-processing model, with three additional models for each of the three classification tasks, including image-quality (e.g., gradable or ungradable), field-of-view (e.g., macula-centered or optic nerve head-centered), and laterality-of-the-eye (e.g., right or left eye). Also included is a cloud-based web application that can output the pre-diagnosis image assessment and a simple binary AD-dementia/non-demented classification based on retinal photographs.

### BACKGROUND OF THE INVENTION

Alzheimer's disease (AD), the most common form of dementia, is a global public health problem.¹ The diagnosis of AD is complex and typically involves expensive and sometimes invasive tests not commonly available beyond highly specialized clinical settings. For example, biomarkers of amyloid-β and phosphorylated tau measured using cerebrospinal fluid (CSF), positron emission tomography (PET) scans, and plasma assays are helpful for AD diagnosis. However, these tests are not suitable for screening possible AD cases in primary care or community settings.^{2,3} Importantly, as new therapeutic efforts focus on early AD treatment,⁴⁻⁶ simple, accessible, and sensitive community-based screening tests would significantly improve population-based strategies to manage AD.

The retina is a highly accessible part of the central nervous system, with common embryology, anatomy, and physiology.⁷ Retinal changes in AD have been shown in histopathological studies of post-mortem specimens.^{8,9} This is further supported by clinical studies showing a range of retinal changes in subjects with AD, such as changes in the retinal vasculature (e.g., vessel caliber and retinopathy signs), the optic nerve, and the retinal nerve fiber layer (RNFL).¹⁰⁻¹⁴ These features can be non-invasively imaged using digital retinal photography, which is now widely available and affordable in primary care optometry and community settings.

Artificial intelligence (AI), particularly deep learning (DL), has been applied to retinal photographs for detecting various ophthalmic diseases (e.g., diabetic retinopathy^{16,17}, optic disc papilledema¹⁸, glaucoma¹⁹, and age-related macular degeneration²⁰). Furthermore, DL approaches can also detect systemic diseases based on retinal photographs (e.g., systemic biomarkers²¹, cardiovascular disease^{22,23}, diabetes²⁴, chronic kidney disease^{24,25}, hepatobiliary diseases²⁶). Nevertheless, the role of DL approaches in detecting patients with AD from retinal photographs has yet to be determined.

In addition, integrating DL algorithms into real-time clinical workflow has been recognized as a priority to realize the significant potential of AI for clinical diagnosis and disease risk stratification²⁷⁻³⁰. However, while many DL algorithms have shown promising results in laboratory and research settings, their performances in real-world clinical settings require further evaluations³¹. A major challenge is that retinal photographs captured from real-world clinical settings can have lower quality than the retinal photographs carefully curated and used specifically in DL algorithms development^{27,32-34}, and thus, the performances of such DL algorithms are less reliable when applied clinically^{27,34}. For example, Abramoff et al. reported while their DL algorithm achieved a sensitivity of 97% in a retrospective dataset under a laboratory setting, the performance dropped to 87.2% in a prospective study conducted in a primary care setting²⁷. In another prospective study conducted by Beede et al.³⁴, about 21% of retinal photographs were unsuitable for DL-based diabetic retinopathy screening because of low image quality. These were likely due to the exclusion of low-quality retinal photographs when training DL algorithms for the eye disease diagnosis^{17,35-38}. As such, the application of these algorithms in real-world clinical settings would require the exclusion of retinal photographs of low image quality to inhibit deterioration of their diagnostic performance³⁹⁻⁴². In addition to image-quality assessment, DL can further provide other useful information such as field-of-view and laterality-of-the-eye before disease diagnosis by subsequent DL processing algorithms. For example, DL algorithms developed for optic disc diseases (e.g., papilledema and glaucoma) should focus on optic disc-centered retinal photographs as it can work less well for macula-centered retinal photographs⁴³⁻⁴⁵.

### BRIEF SUMMARY OF THE INVENTION

Embodiments of the subject invention provide an AI-aided classification system for AD screening from retinal photographs, which includes a DL-based pre-diagnosis module for image assessment and a DL-based AD classification module with additional heatmaps for visualization. Provided embodiments of the AI system can output a pre-diagnosis image assessment (e.g., the image-quality, field-of-view, and laterality-of-the-eye) and a simple binary AD-dementia/non-demented classification based on retinal photographs. Certain embodiments can add a complimentary risk profiling tool for AD and assist physicians to identify asymptomatic individuals who are more likely to have AD in the community. Higher-risk individuals can then benefit from selective referral for more intensive and specific examinations (e.g., PET imaging, plasma assays for amyloid-β and phosphorylated tau) at highly specialized clinics for facilitating early AD diagnosis and allowing the individuals to take prevention measures, such as lifestyle modification and control of risk factors.

There is increasing evidence that a range of retinal features identified from retinal photographs is associated with AD. DL has been shown to have significant potential for eye disease detection and screening on retinal photographs in different clinical settings, particularly in primary care. Nevertheless, the role of DL approaches in detecting patients with AD from retinal photographs has yet to be determined. Besides, integrating DL algorithms into real-time clinical workflow is a priority to realize the significant potential of AI for clinical diagnosis and disease risk stratification. In addition, an automated pre-diagnosis image assessment is advantageous to streamline the application of the developed DL algorithms.

Embodiments of the subject invention provide an AI-aided classification system for AD screening from retinal photographs, which includes a DL-based pre-diagnosis module for image assessment and a DL-based AD classification module with additional heatmaps for visualization. For AD classification, embodiments provide three kinds of DL models that predict the AD-dementia/non-demented probabilities from three directions with both eyes' four images (Direction-1), both eyes' four images combining demographical information (Direction-2), and a single eye's two images (Direction-3), respectively. For the pre-diagnosis image assessment, it consists of one image pre-processing model, with three additional classification models for classifying image-quality (e.g., gradable or ungradable), field-of-view (e.g., macula-centered or optic nerve head-centered), and laterality-of-the-eye (e.g., right or left eye).

Embodiments provide a cloud-based web application and can output a pre-diagnosis image assessment (e.g., image-quality, field-of-view, and laterality-of-the-eye) and a simple binary AD-dementia/non-demented classification based on retinal photographs. The results can add a complimentary risk profiling tool for AD and assist physicians to identify asymptomatic individuals who are more likely to have AD in the community. Higher-risk individuals can then benefit from selective referral for more intensive and specific examinations (e.g., PET imaging, plasma assays for amyloid-β and phosphorylated tau) at highly specialized clinics for facilitating early AD diagnosis and allowing the individuals to take prevention measures, such as lifestyle modification and control of risk factors. It is contemplated that certain embodiments will be incorporated into retinal photography devices for automated image analysis and AD screening in different scenarios.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically represents a sequence diagram of the pre-diagnosis module according to an embodiment of the subject invention. The module will evaluate the image quality at first (e.g., gradable or ungradable), and then identify the laterality-of-the-eye (e.g., right or left eye) and field-of-view (e.g., macula-centered or optic nerve head-centered) for gradable images.

FIGS. 2A-2C Show the core deep learning algorithms of the classification module according to an embodiment of the subject invention, which predict the Alzheimer's disease (AD)-dementia/non-demented probabilities from three directions: 2A shows Direction-1 using both eyes' four images (e.g., macula-centered and optic nerve head-entered images from both eyes); 2B shows Direction-2 using both eyes' four images (e.g., macula-centered and optic nerve head-entered images from both eyes) combining demographical information (e.g. age, gender, yes/no hypertension, yes/no diabetes); 2C shows Direction-3 using single eye's two images (e.g., macula-centered and optic nerve head-entered images from only one eye).

FIGS. 3A-3B Show procedures for AD screening by an embodiment of the subject invention: 3A shows the workflow including retina photography, pre-diagnosis assessment, and AD-dementia/non-demented prediction; 3B shows the screening scenario. The invention can potentially screen out participants with moderate or high risk of AD-dementia and refer these participants to further confirmatory investigations (e.g., PET imaging, cerebrospinal fluid) at specialized clinics, implementation of potential preventive interventions (e.g., lifestyle modifications), or recruitment into clinical trials.

FIGS. 4A-4D Show example heatmaps of truly detected cases by the Direction-1 model according to an embodiment of the subject invention: 4A and 4B show heatmaps of truly classified retinal photographs in two cases with AD-dementia; 4C and 4D show heatmaps of truly classified retinal photographs in two non-demented cases. The red-orange-colored area represented the most discriminative area for the model to classify AD. As illustrated, for truly classified cases, the model tends to focus on the inferior area in the retina, covering both the retinal nerve fiber layer and retinal vessels.

FIGS. 5A-5C Show the operation of image uploading by end users and AI output of image assessment and AD classification according to an embodiment of the subject invention. The end user can upload a folder (or optionally, another data structure) with four retinal photographs (e.g., macula-centered and optic nerve head-entered images from both eyes) to the web interface (5A and 5B); the provided system can output the pre-diagnosis image assessments and the results of AD-dementia (5C). The pre-diagnosis output includes the image quality (e.g., gradable or ungradable), the laterality-of-the-eye (e.g., right or left eye), and field-of-view (e.g., macula-centered or optic nerve head-centered); the AD-dementia output includes “Alzheimer's disease dementia” or “non-demented”.

FIG. 6 schematically represents the ground-truth labeling and the data distribution of the primary and external datasets according to an embodiment of the subject invention.

FIGS. 7A-7D Illustrate examples of retinal photographs with different levels of image quality according to embodiments of the subject invention. A gradable retinal photograph had to fulfill both of the following criteria: (1) less than 25% of the peripheral area of the retina was unobservable due to artifacts, and (2) the center region of the retina had no significant artifacts. (7A, 7B) Gradable and absence of any artifacts; (7C) ungradable as more than 25% of the peripheral area of the retina was unobservable due to the presence of eyelid; (7D) ungradable due to the presence of significant artifacts in the center region.

FIGS. 8A-8E Illustrate examples of retinal photographs with different fields of view according to embodiments of the subject invention: (8A) macula-centered; (8B) optic-disc-centered; (8C-8E) off-centered. Horizontal and vertical auxiliary lines were added to locate the center region of the retinal photograph, which was bounded by a green circle. The center region of a retinal photograph is defined as the circular region of radius (in pixels) with the largest integer not greater than one-tenth the width of the image at the center of the image. A green circle bounded the center region of the retinal photograph.

FIG. 9 Shows a pipeline of retinal photograph normalization according to an embodiment of the subject invention. Each retinal photograph undergoes several steps for normalization including region of interest (ROI) cropping, background subtraction with median filters, removal of the illuminated rim and stacking normalized images image from step 1 and step 3 and original image, and then output with a 6-channel fused retinal photograph.

FIG. 10 Shows an overview of the Basic Model according to an embodiment of the subject invention. In Phase 1, the basic model can extract features from optic nerve head-centered or macula-centered images individually. A domain-specific batch normalization block provides unsupervised domain adaptation (the dotted blue box). The batch normalization is split into two batch normalizations (BN1 and BN2) for source and target domain images, respectively. All the remaining layers are shared through the network. The distribution of the optic nerve head-centered or macula-centered images was 1:1. The features were then integrated by the fusion network for further disease classification.

FIG. 11 Shows the performance comparison of the proposed bilateral model according to an embodiment of the subject invention and the cognitive screening testing using the Hong Kong version of MoCA for Alzheimer's disease detection in the community-based cohort (i.e., External-5 or Testing-5).

FIGS. 12A-12C Show an overview of three proposed deep learning models according to an embodiment of the subject invention. The models each, respectively, provide a domain-specific batch normalization block for unsupervised domain adaptation. All the remaining layers can be shared through the network. (12A) The bilateral model feeds four retinal photographs, including optic nerve head-centered and macula-centered photographs of right and left eye for the classification of Alzheimer's disease-dementia and no dementia. (12B) The unilateral model fuses the information from both optic nerve head-centered and macula-centered images in each eye for the Alzheimer's disease-dementia prediction. (12C) The hybrid model is based on the provided bilateral model, by integrating demographic information into the deep models using bilinear transformation.

FIGS. 13A-13C Show Table 3-1: Characteristics of the primary training and validation, and testing datasets, from Example 3.

FIGS. 14A-14B Shows Table 3-2: The participant-level performance of the deep learning bilateral model and unilateral model in the internal validation and the testing datasets, from Example 3.

FIG. 15 Shows Table 3-3: The participant-level performance of the deep learning-based model stratified by eye disease and diabetic mellitus in the testing datasets with amyloid-PET imaging, from Example 3.

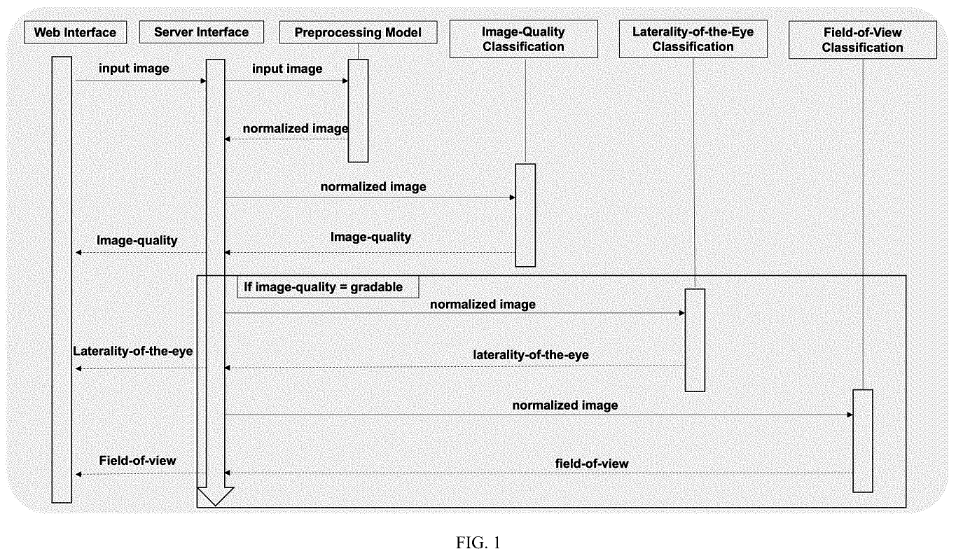

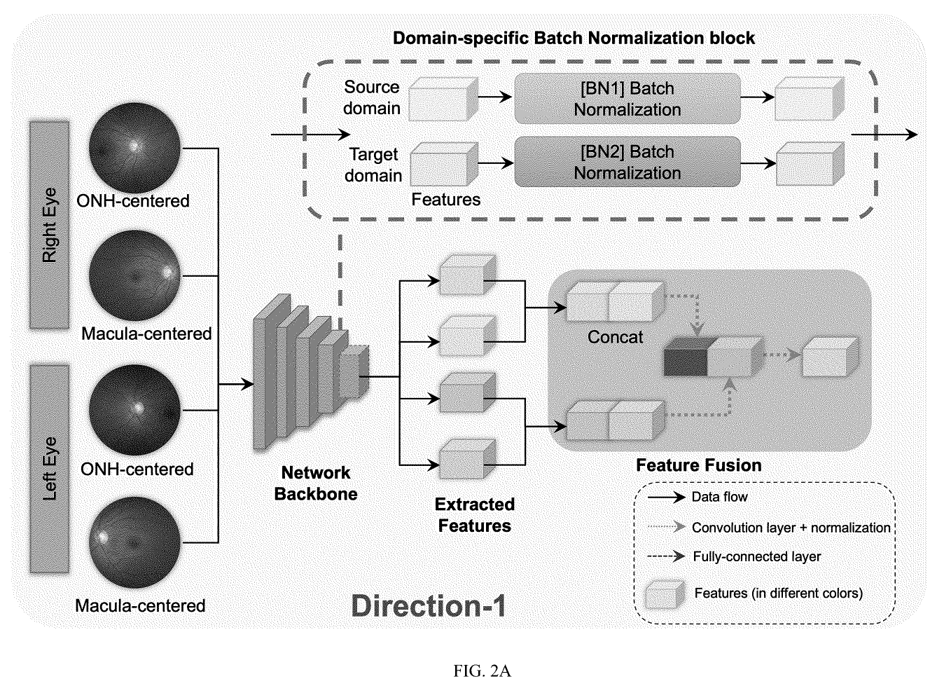

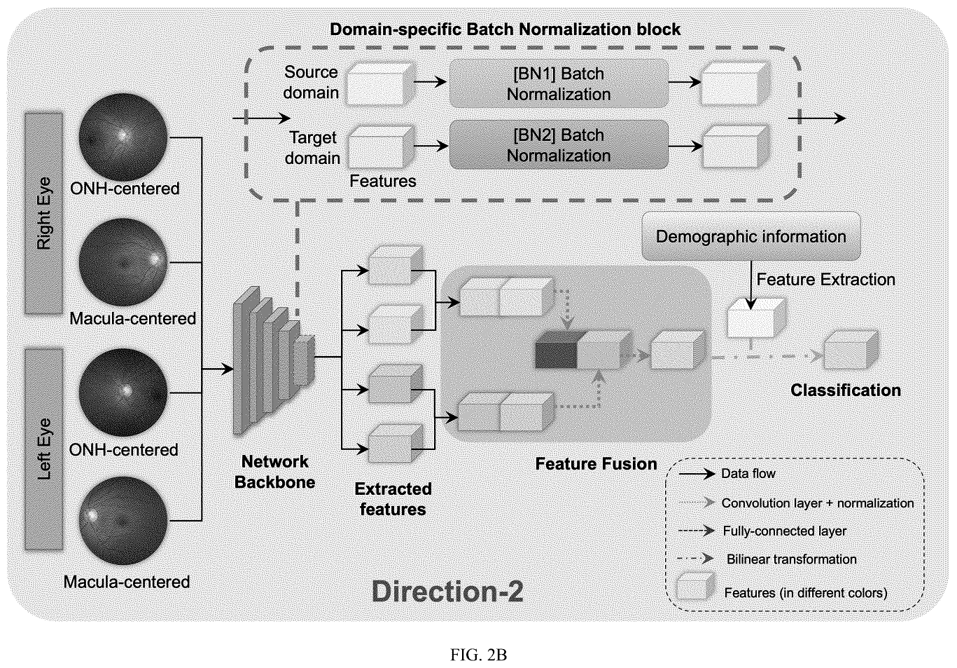

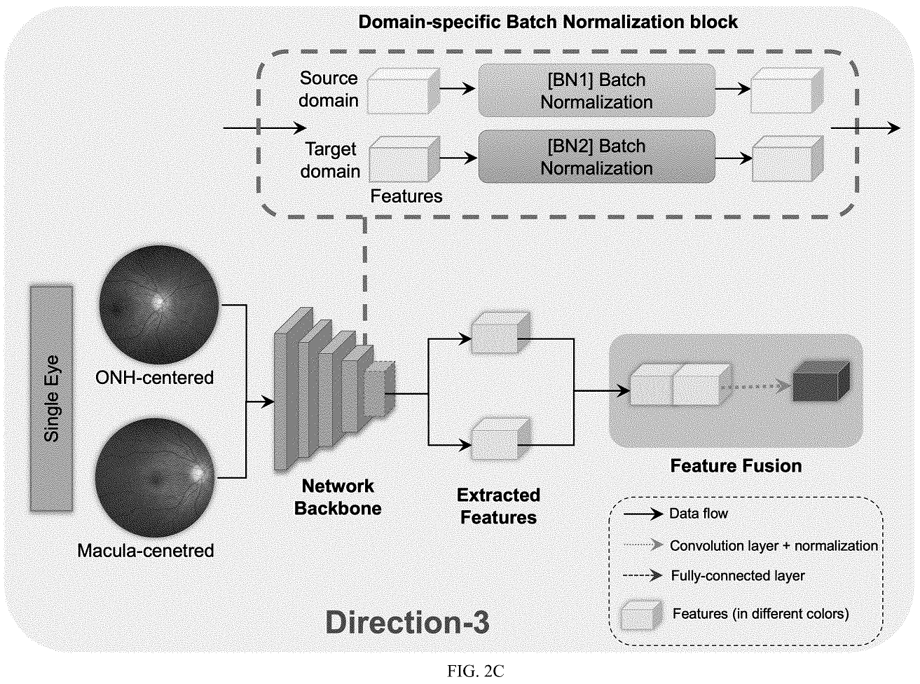

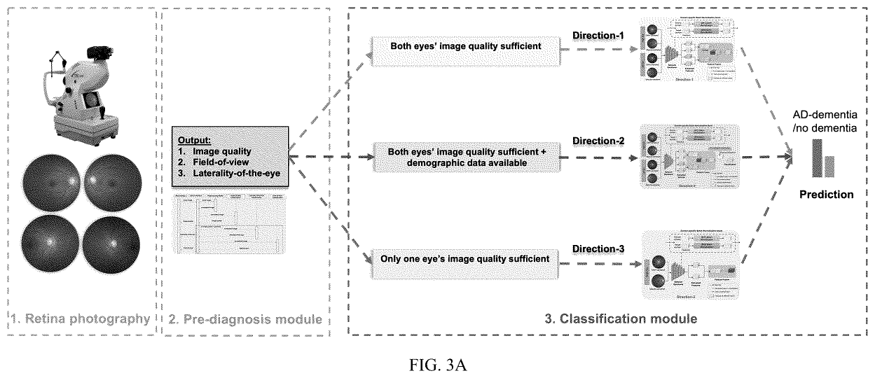

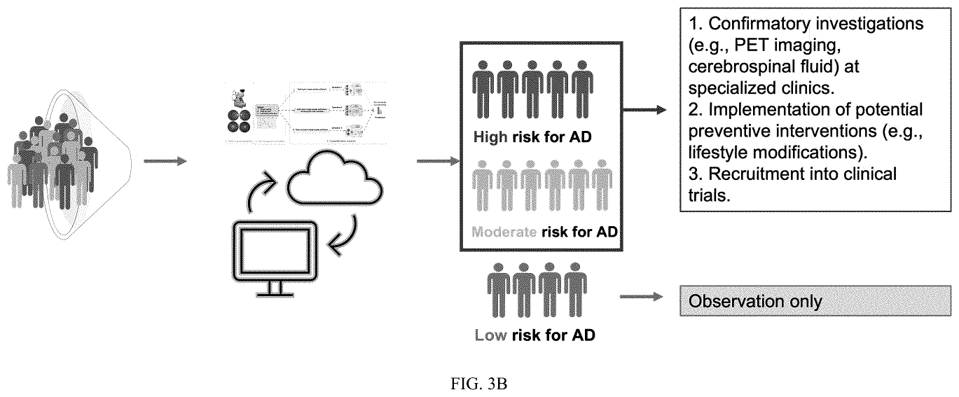

### DETAILED DISCLOSURE OF THE INVENTION

Embodiments of the subject invention provide an AD classification deep-learning module. Certain embodiments provide up to three kinds of DL models which predict the AD-dementia/non-demented probabilities from three directions with both eyes' four images (Direction-1), both eyes' four images combining demographical information (Direction-2), and single eye's two images (Direction-3), respectively.

Embodiments advantageously applied EfficientNet-b2⁴⁶ as the backbone for feature extractor and then a DL model was designed to integrate AD-related features from four retinal photographs for each study subject (e.g., both optic nerve head-and macula-centered fields from both eyes) (Direction-1). This model outputted subject-level detection results (e.g., AD-dementia or non-demented) accounting for AD features from both eyes' images. Second, on top of Direction-1, embodiments further trained another DL model which can additionally consider risk factors of AD (e.g., the demographical information including age, gender, presence or absence of hypertension and diabetes) (Direction-2). Finally, embodiments developed a DL model for single eye analysis as individuals can have an ungradable retinal photograph from one eye (e.g., due to severe cataract) (Direction-3).

For each AD classification model, embodiments adopted unsupervised domain adaptation with domain-specific batch normalization to address the issue of data heterogeneity and domain shift problems from different study cohorts and to improve the model generalizability. Unsupervised domain adaptation is a type of learning framework that can transfer knowledge learned from a larger number of annotated training data in the source domains to target domains with unlabeled data only. Certain embodiments provide domain-specific batch normalization as a building block for deep neural networks where the source domain and the target domain datasets can have their own separate batch normalization layer for training and extraction of hyper-parameters. This design serves to address characteristics specific to each domain that are not compatible within a single model, while retaining domain-invariant information that is common to all domains. In certain embodiments, the labelled source domain dataset was first used for training in a supervised way to generate an unsupervised domain adaption network. This unsupervised domain adaptation network was then used to generate pseudo-labels for unlabeled data in the target domain. The final classification network was subsequently trained with full supervision using labelled data from source domain and pseudo-label from target domains. Through the fusion of the domain-independent and domain-dependent knowledge learning, the provided DL models can transfer discriminative features from the labelled source domain to the unlabeled target domain (e.g., domain adaptation) and improve the classification performance on the target domain. Due to the limitation of the domain adaptation-based method, embodiments trained one model for each external dataset to obtain the prior information of unlabeled external datasets.

To better understand discriminative features between AD-dementia subjects and no dementia subjects, embodiments advantageously applied Gradient-weighted Class Activation Mapping (Grad-CAM)⁴⁷ to visualize these features.

### MATERIALS AND METHODS

Alzheimer's disease (AD), the most common form of dementia, is a major public health and clinical challenge globally, causing a significant socioeconomic burden worldwide. Although cerebrospinal fluid (CSF) biomarkers and recent novel biomarkers include positron emission tomography (PET) scans and plasma assays for amyloid-β and phosphorylated tau showed great promise for aiding AD detection, particularly early-stage AD, these tests (e.g., CSF, PET) are not suitable for screening in routine clinical settings or communities. As early AD is now the focus of new therapeutic efforts and treatment of AD is possible, a more widely accessible screening system to identify individuals in the community, who are then referred to neurology clinics for more intensive and specific examinations to confirm AD would aid the management of AD.

The retina, a neurosensory layered tissue lining the back of the eye, has long been considered as a proxy measure to study disorders in the central nervous system (CNS), as it is an accessible extension of the brain in terms of embryology, anatomy, and physiology. Evidence of retinal pathology in AD has been shown in histopathological studies of postmortem specimens. Meanwhile, the retinal vasculature, optic nerve head (ONH), and retinal nerve fiber layer (RNFL) can be captured and assessed using retinal photography effectively and non-invasively at a relatively low cost, making it a potential ideal tool for community screening for AD. Accumulating data have shown that specific retinal features measured from retinal photographs are associated with AD, including RNFL loss, vessel caliber, vessel tortuosity, vessel fractal dimension, and retinopathy signs. A simple whole retina score, in principle, would be even more useful as a clinical screening tool for AD.

Artificial intelligence (AI), particularly deep learning (DL), can provide such a solution to facilitate the application of retinal photography for the screening of AD. DL allows an algorithm to appreciate and extract the inherent non-obvious features from training images necessary for accurate discrimination based on examples, without the need for manual engineering of discriminating features. DL has been applied in the assessment of retinal photographs for the detection of various ophthalmic diseases, including diabetic retinopathy, glaucoma, and age-related macular degeneration. However, the possibility of identifying AD from retinal photographs alone using the DL approach has yet to be determined.

Embodiments of the subject invention provide a novel AI-aided classification system for AD screening from retinal photographs with additional pre-diagnosis image assessment. Embodiments exhibit several advantages and improvements, including the following:
1) Embodiments integrate retinal photograph assessment of the image-quality, field-of-view, and laterality-of-the-eye into a single pre-diagnosis module.
2) Embodiments integrate different DL models for AD classification from retinal photographs in three directions for different implementation scenarios.
3) Embodiments provide an AD classification module comprising two advanced DL techniques (e.g., unsupervised domain adaptation and feature fusion), to address two significant challenges: (a) data distribution discrepancy between training/validation and testing datasets, and (b) the integration from multiple ONH- and macula-centered retinal photographs from both eyes. With this advanced DL architecture in certain embodiments, analysis can be easily transferrable to a new center without the need to re-develop a new DL model from scratch. Retrospective data can be collected firstly from this specific center for unsupervised domain adaptation, and refinement can then be subsequently performed for updating the DL model.
4) Embodiments provide a cloud-based web application to conduct pre-diagnosis image assessment and AD classification. Embodiments also contain a by-pass function to allow manual justification on the AI-based pre-diagnosis image assessment results and ensure the success of downstream tasks for AD classification.

Embodiments provide at least two potential implementations in clinical practice:
1) Embodiments can accommodate algorithm use for AD screening by pre-diagnosis image assessment and ensuring the gradability of retinal photographs by providing an immediate onsite assessment of image quality. This can allow retaking of retinal photographs, if necessary, of subjects within the same visit and also reduce the expertise required in collecting retinal photographs. In addition, the automatic identification of the field of view and laterality of the eye can also minimize mislabeling errors and provide more information to facilitate diagnosis.
2) Embodiments can be integrated into a comprehensive clinical pipeline for use in the community and facilitate a more widespread use of AI-aided systems to assist low-cost AD screening and lead to improved AD prevention.

Embodiments of the subject invention address the technical problem of detecting and screening for early-stage AD or dementia being expensive, needing excessive human processing, not being suitable for screening large populations, and requiring invasive methods.

This problem is addressed by providing digital image processing with enhanced AI, in which a deep learning method applying a combination of advanced techniques is utilized to categorize images based on the classification given during the learning process.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

Embodiment 1. An artificial intelligence (AI)-aided classification system for Alzheimer's disease (AD) screening from a source dataset comprising retinal photographs, the system comprising a deep learning (DL) model created by a process comprising:
a) application of one or more data pre-processing methods and one or more on-the-fly data augmentation methods to normalize the retinal photographs including region of interest (ROI) cropping,
b) background subtraction with median filters, and
c) removal of an illuminated rim and a stacking rim to create a multitude of cropped images to generate a 6-channel input for training the DL model.

Embodiment 2. The system according to Embodiment 1, the DL model comprising a bilateral model, a hybrid model, and a unilateral model.

Embodiment 3. The system according to Embodiment 2, wherein:
the bilateral model is trained primarily on both eyes' four images;
the hybrid model is trained primarily on both eyes' four images combing demographic information; and
the unilateral model is trained primarily on single eye's two images.

Embodiment 4. The system according to Embodiment 1, the source dataset comprising retinal photographs and demographic information derived from different centers, and the DL model comprising a feature fusion module to integrate features captured from the different centers.

Embodiment 5. The system according to Embodiment 1, the DL model comprising demographic information integrated with both eyes' four images from one subject; and demographic information integrated by bilinear transformation.

Embodiment 6. The system according to Embodiment 1, the DL model comprising EfficientNet-b2 as a backbone to extract features.

Embodiment 7. The system according to Embodiment 6, the DL model comprising a domain adaptation technique to deal with dataset discrepancies.

Embodiment 8. The system according to Embodiment 1, the DL model created by a process comprising two stages.

Embodiment 9. The system according to Embodiment 8, the two stages comprising:
a first stage, trained with supervised learning on the source dataset with image-level annotations; and
a second stage, introducing a domain adaptation method by estimating the pseudo labels for the retinal photographs from the source dataset using a domain-specific batch normalization technique.

Embodiment 10. The system according to Embodiment 9, wherein images from the source and target domains were fed into separate batch normalization layers in each of the first stage and the second stage, respectively.

Embodiment 11. The system according to Embodiment 10, comprising an imbalance of data between a first class with more data and a second class with less data, and comprising an over sampling for the second class.

Embodiment 12. The system according to Embodiment 11, comprising a training objective function utilizing both source dataset and target domain images.

Embodiment 13. The system according to Embodiment 12, comprising generation of heatmaps to show the significant locations which are related to the AD with a Gradient-weighted Class Activation method.

Embodiment 14. The system according to Embodiment 13, comprising pre-diagnosis image assessment and AD binary classification by a cloud-based web application.

Embodiment 15. A method for creating an artificial intelligence (AI)-aided classification system comprising a deep learning (DL) model for Alzheimer's disease (AD) screening from a source domain comprising retinal photographs, the method comprising:
a) applying one or more data pre-processing methods and one or more on-the-fly data augmentation methods to normalize the retinal photographs, including region of interest (ROI) cropping,
b) subtracting background with median filters, and
c) removing an illuminated rim and a stacking rim to create a multitude of cropped images to generate a 6-channel input for training the DL model;
d) training a bilateral model primarily on both eyes' four images;
e) training a hybrid model primarily on both eyes' four images combing demographic information;
f) training a unilateral model primarily on single eye's two images; and
g) applying a feature fusion module to integrate features captured from different centers; and
h) mapping from the source domain to create a target domain.

Embodiment 16. The method according to Embodiment 15, comprising:
i) integrating demographic information with both eyes' four images from one subject; and
j) integrating demographic information by bilinear transformation.

Embodiment 17. The method according to Embodiment 16, comprising:
k) extracting features from the source domain using EfficientNet-b2 as a backbone;
l) applying a domain adaptation technique to deal with dataset discrepancies;
m) conducting a first stage, trained with supervised learning on the source dataset with image-level annotations wherein images from the source domain and from the target domain were fed into separate batch normalization layers;
n) conducting a second stage, introducing a domain adaptation method by estimating pseudo labels for the retinal photographs from the target domain using domain-specific batch normalization technique wherein images from the source domain and from the target domain, respectively, are fed into separate batch normalization layers;
o) identifying an imbalance of data between a first class with more data and a second class with less data;
p) applying an over sampling for the second class;
q) applying a training objective function utilizing both source domain images and target domain images;
r) generating heatmaps to show the significant locations which are related to the AD with a Gradient-weighted Class Activation method; and
s) providing both a pre-diagnosis image assessment and an AD binary classification by a cloud-based web application.

Embodiment 18. An artificial intelligence (AI)-aided classification system for Alzheimer's disease (AD) screening from a source dataset comprising retinal photographs, the system comprising a deep learning (DL) model, the system created by a process comprising:
a) applying one or more data pre-processing methods and one or more on-the-fly data augmentation methods to normalize the retinal photographs including region of interest (ROI) cropping,
b) subtracting background with median filters, and
c) removing an illuminated rim and a stacking rim to create a multitude of cropped images to generate a 6-channel input for training the DL model;
d) training a bilateral model primarily on both eyes' four images;
e) training a hybrid model primarily on both eyes' four images combining demographic information;
f) training a unilateral model primarily on single eye's two images;
g) applying a feature fusion module to integrate the features captured from different centers;
h) training the DL model on demographic information integrated with both eyes' four images from one subject;
i) training the DL model on demographic information integrated by bilinear transformation;
j) extracting features using EfficientNet-b2 as a backbone; and
k) applying a domain adaptation technique to resolve dataset discrepancies.

Embodiment 19. The system according to Embodiment 18, wherein the DL model is created by a process comprising two stages, the two stages comprising:
a) conducting a first stage, trained with supervised learning on the source dataset with image-level annotations to create a target domain comprising the retinal images; and
b) conducting a second stage, introducing a domain adaptation method by estimating pseudo labels for the retinal photographs from the target domain using domain-specific batch normalization technique;
   wherein images from the source dataset and from the target domain were fed into separate batch normalization layers in each of the first stage and the second stage, respectively; and
   wherein an imbalance of data between a first class with more data and a second class with less data was balanced with over sampling for the second class.

Embodiment 20. The system according to Embodiment 19, the system created by a process comprising:
l) applying a training objective function utilizing both source dataset and target domain images;
m) generating heatmaps to show the significant locations which are related to the AD with a Gradient-weighted Class Activation method; and
n) providing both pre-diagnosis image assessment and AD binary classification by a cloud-based web application.

It should be understood that the examples and embodiments described herein are for illustrative purposes only and that various modifications or changes in light thereof will be suggested to persons skilled in the art and are to be included within the spirit and purview of this application and the scope of the appended claims.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Model Creation

A first exemplary embodiment provided a pre-diagnosis deep-learning module consisting of (in alternative embodiments, either comprising or consisting essentially of) one pre-processing model, with three additional models for each of the three classification tasks. As different retinal cameras captured retinal photographs with different image resolutions and degrees of view, image pre-processing was first performed to normalize the inputs to similar conditions. Image normalization was performed using the preprocessing module to standardize inputs to similar conditions. Data balancing and data augmentation were applied on the fly. During training, the pre-trained ImageNet weights were used for initial weighting. Furthermore, these assessment modules were converted into TFLite models to reduce latency inference. For all tasks, input data was randomly augmented with (−0.3, 0.3) brightness adjustment, (−0.5, 0.5) contrast adjustment, (−0.5, 0.5) saturation adjustment, (−0.1, 0.1) hue adjustment, along with 60 degrees of random rotation, 20% random translation, 10% scaling and 5 degrees of shearing. All images were augmented channel-wise with means of (0.485, 0.456, 0.406) and standard deviations of (0.229, 0.224, 0.225).

Recognizing that the tasks are different and models tend to learn different features, the inventors used EfficientNet-B0⁴⁸ for the image-quality and the field-of-view tasks, and MobileNetV2⁴⁹ for the laterality-of-the-eye task, respectively, to make use of advantages in different architectures. All the retinal photographs were used to train the image-quality assessment model. After excluding ungradable retinal photographs and off-centered retinal photographs, only gradable macula-centered and optic disc-centered retinal photographs were used to train the DL algorithms for field-of-view and laterality-of-the-eye assessments.

The inventors provided a cloud-based web application, advantageously integrating the whole process of data pre-processing, data analysis, and data output of image assessment, AD classification. The application was composed under the service-oriented-architecture (SOA) protocol, which facilitates case of maintenance. From the user perspective, no additional operation was required apart from uploading retinal photographs. The cloud-based web application also contains a by-pass function to allow manual justification on the AI-based image assessment results and ensure the success of downstream task for AD classification.

This exemplary embodiment provides a novel AI-aided classification system for AD screening from retinal photographs with additional pre-diagnosis image assessment, including the following advantageous elements:
1) Integrated retinal photograph assessment of image quality, field of view, and laterality of the eye into a single pre-diagnosis module.
2) Integrated different DL models for AD classification from retinal photographs in three directions for different implementation scenarios.
3) The AD classification module was developed with two advanced DL techniques (e.g., unsupervised domain adaptation and feature fusion), to address two significant challenges: 1) data distribution discrepancy between training/validation and testing datasets, and 2) the integration from multiple ONH- and macula-centered retinal photographs from both eyes. With this advanced DL architecture, the model can be easily transferrable to a new center without the need of re-developing a new DL model from scratch. Retrospective data can be collected firstly from this specific center for unsupervised domain adaptation, and refinement can then be subsequently performed for updating the DL model.
4) A novel cloud-based web application to conduct pre-diagnosis image assessment and AD classification. The cloud-based web application also contains a by-pass function to allow manual justification on the AI-based image assessment results and ensure the success of downstream task for AD classification.

Embodiments provide at least two potential implementations in clinical practice:
1) Embodiments can accommodate algorithm use for AD screening by pre-diagnosis image assessment and ensuring the gradability of retinal photographs by providing an immediate onsite assessment of image quality. This can allow retaking of retinal photographs, if necessary, of subjects within the same visit and also reduce the expertise required in collecting retinal photographs. In addition, the automatic identification of field of view and laterality of the eye can also minimize errors from mislabeling and provide more information to facilitate diagnosis.
2) Embodiments can be integrated into a comprehensive clinical pipeline in the community and facilitate a more widespread use of AI-aided system to assist low-cost AD screening and lead to improved AD prevention.

Commercial relevance and market potential for one market was estimated by using the following formula:

$$MP = N \times MS \times P \times Q$$

Where MP represents the market potential; N represents the total potential customers in Hong Kong; MS represents market share, which is the percent of consumers buying the AD screening service; P represents selling price for the screening service for each costumer, and Q represents average annual consumption. The inventors defined the potential customers as the elderly community (subjects >65 years old) in Hong Kong. Referring to statistical reports, there were around 1.3 million people aged 65 years old or above living in domestic household in Hong Kong in 2022 (https://www.statista.com/statistics/962290/hong-kong-elderly-population-in-domestic-households-by-age-group/). Regarding the market share, currently there is no existing AI-aided classification system for AD screening from retinal photographs with additional pre-diagnosis image assessment in the market. Therefore, the inventors estimated a 40% percent share of the market since the business is new. For the “P” selling price, referring to the price of retinal photography in the CUHK eye center, the inventors estimate the selling price for each subject for the screening service can be 300 HKD. A study showed rescreening every 5 years can reduce the prevalence of dementia due to AD by 50%.⁵⁰ Therefore, the estimated market potential will be:

$$M P = 1 , 300 , 000 \times 40 \% \times 300 \times 0.2 = 31 , 200 , 000 HKD ( US\$ 3 , 974 , 814 )$$

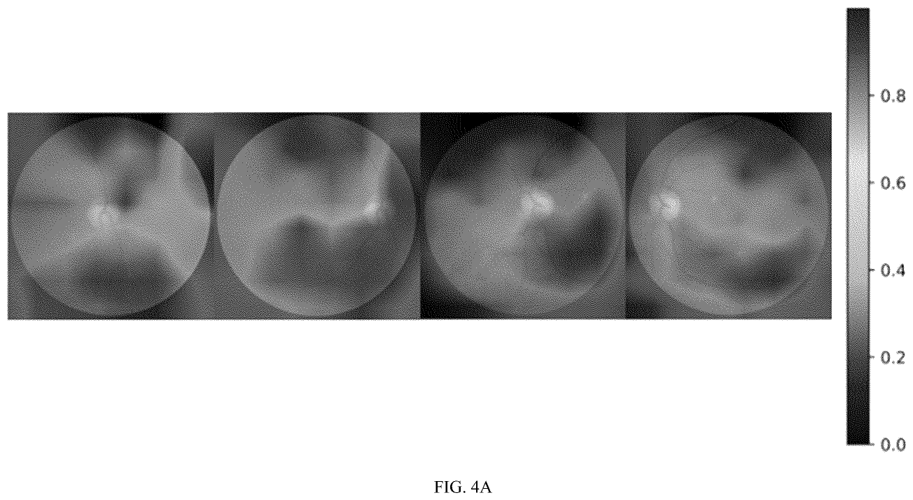

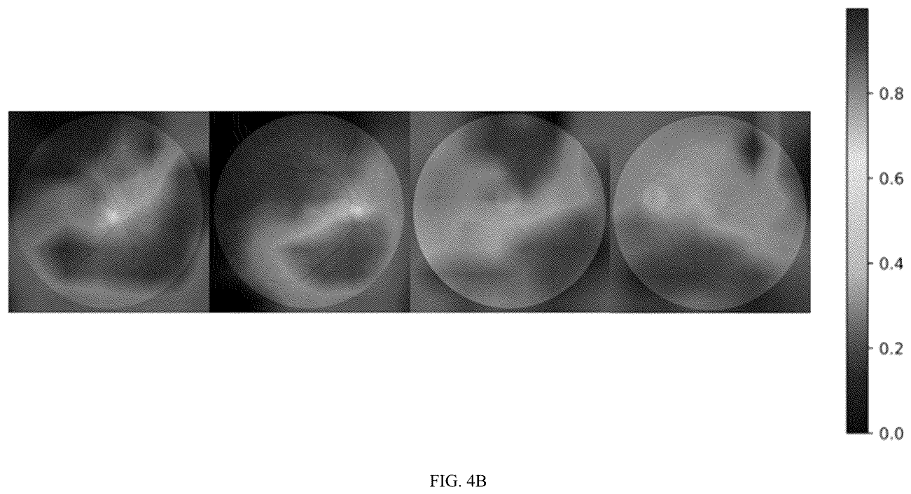

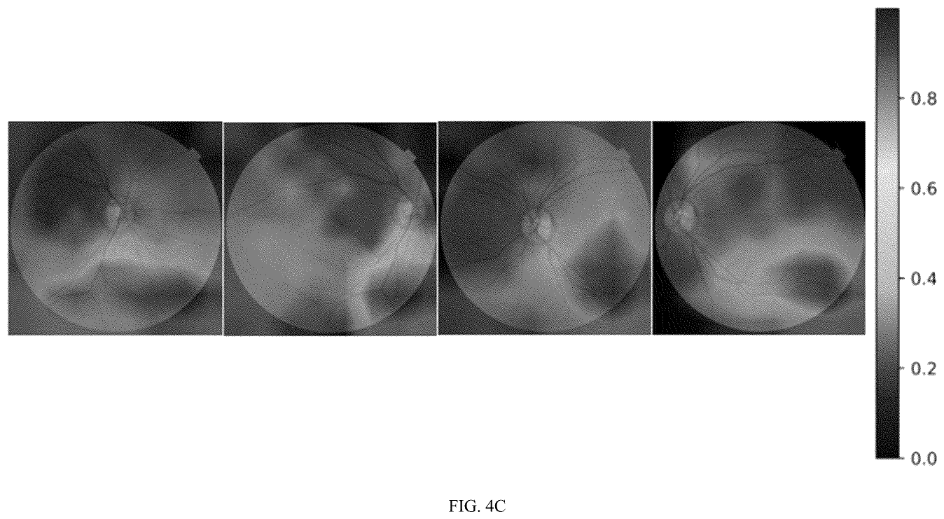

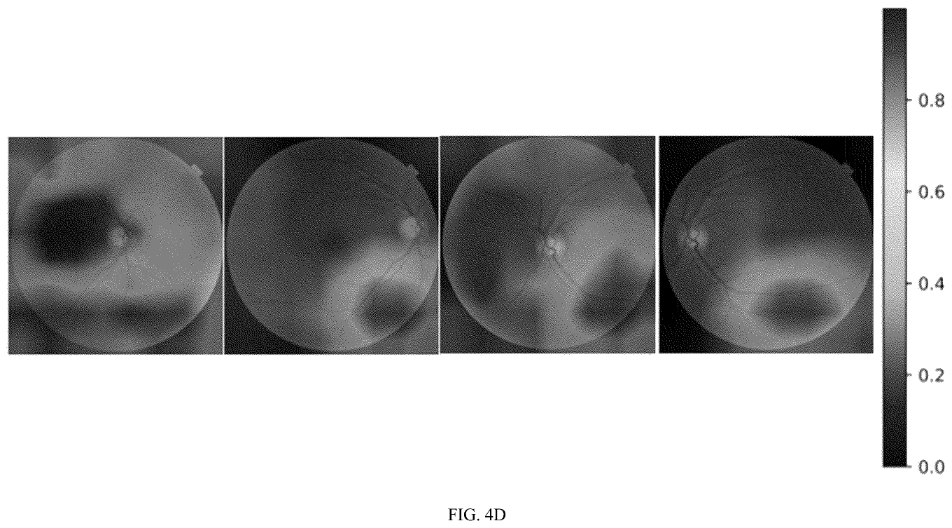

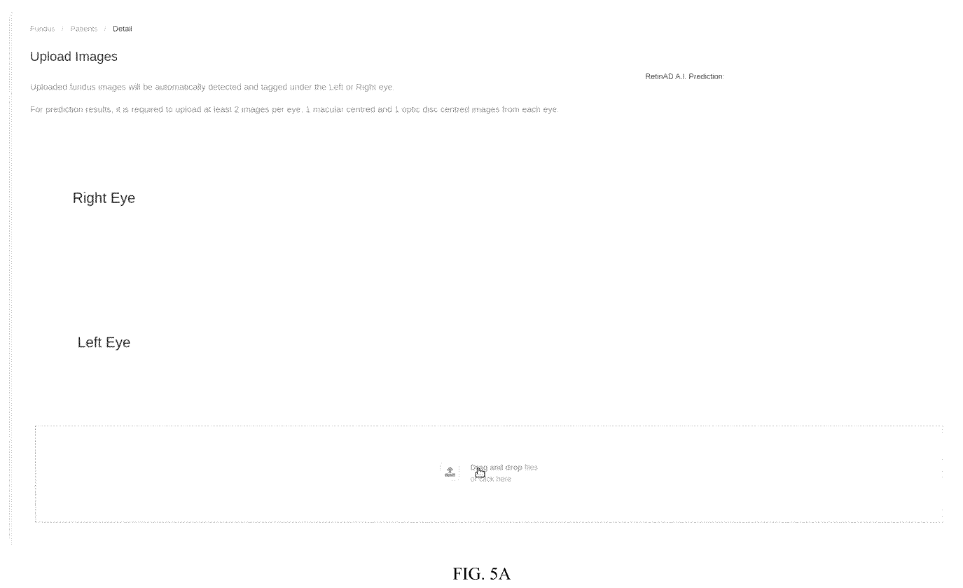

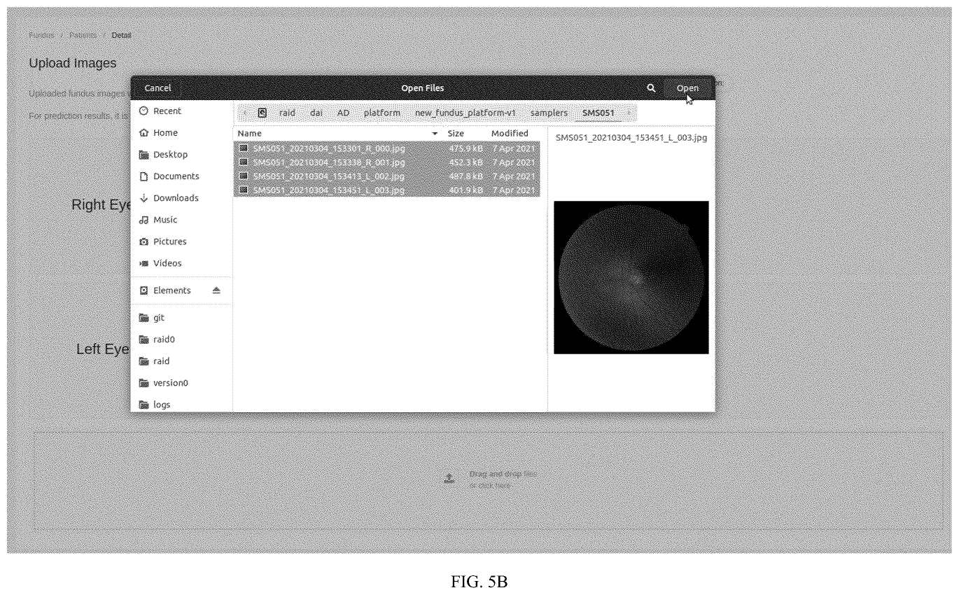

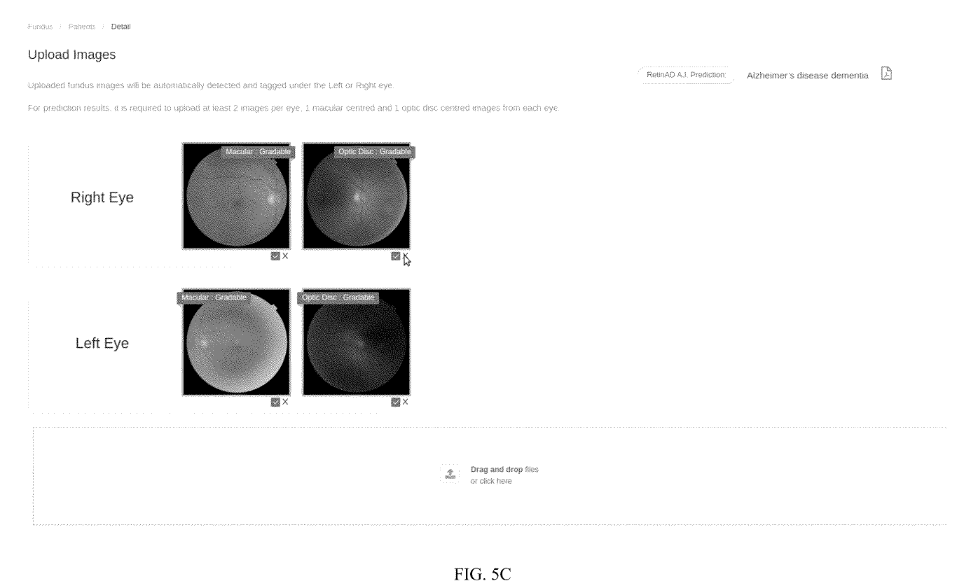

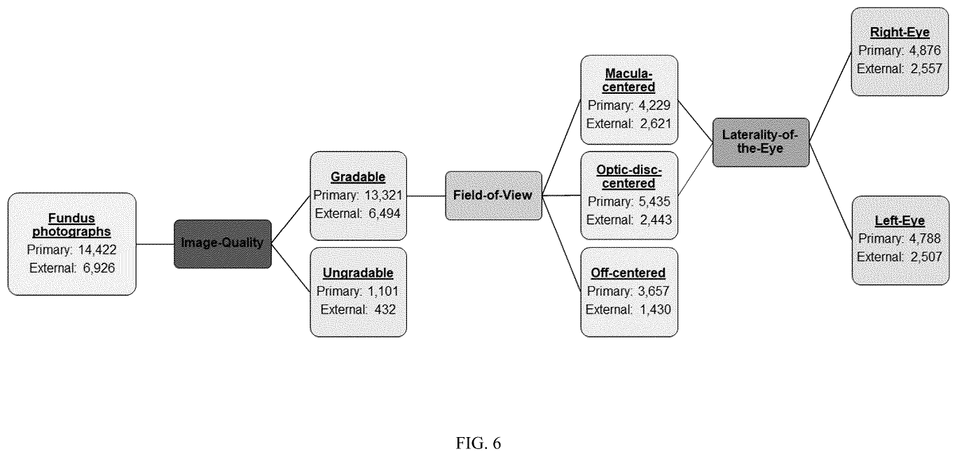

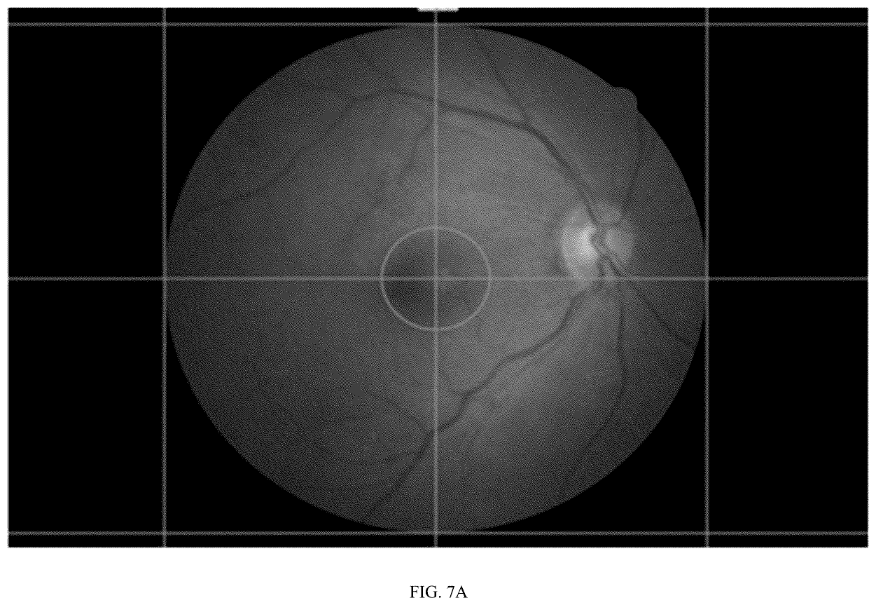

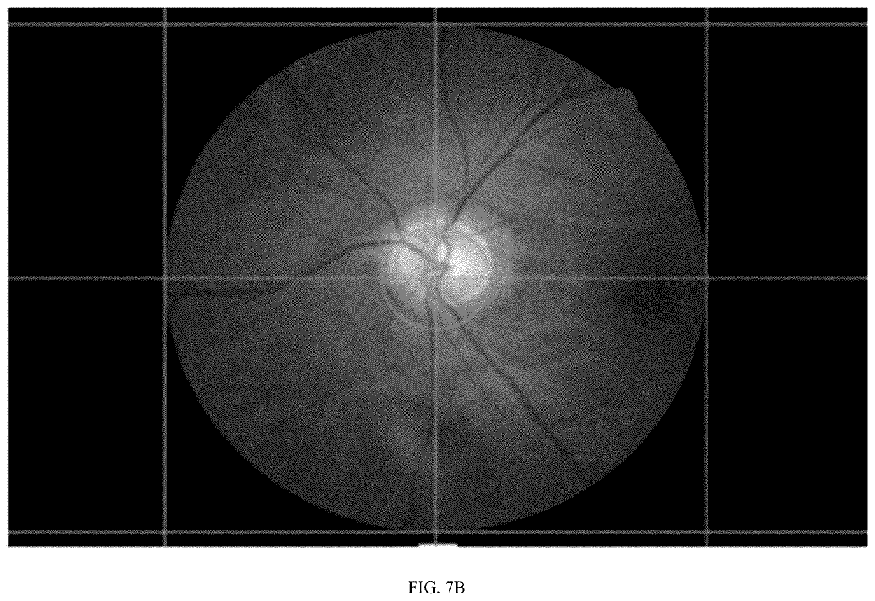

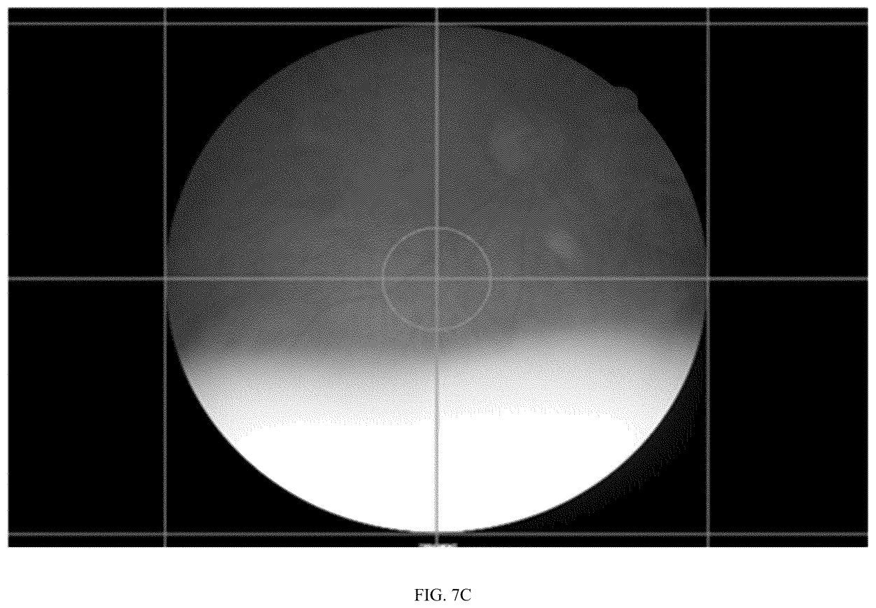

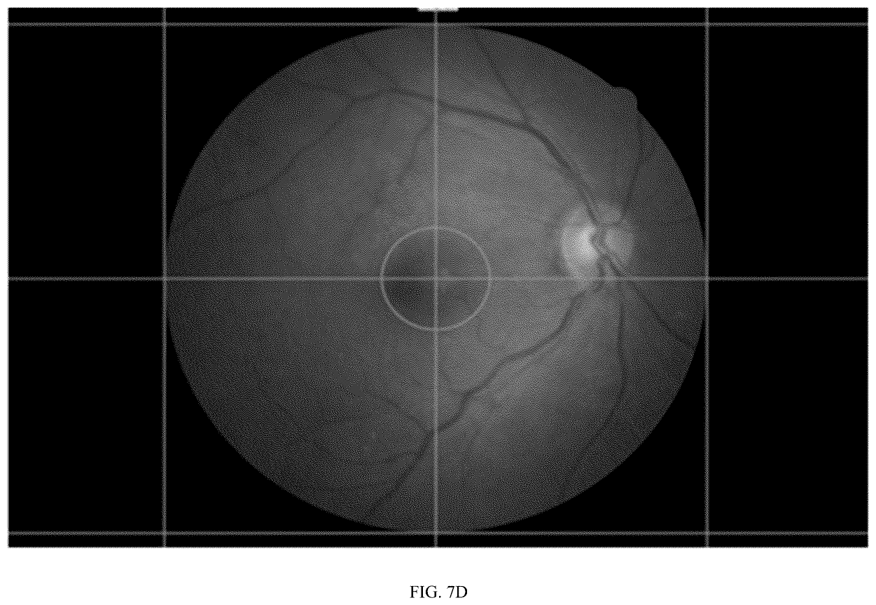

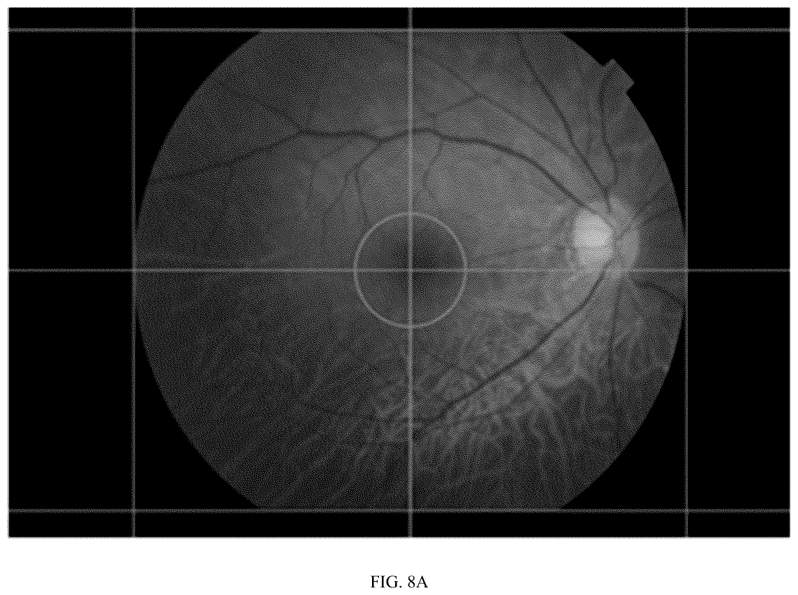

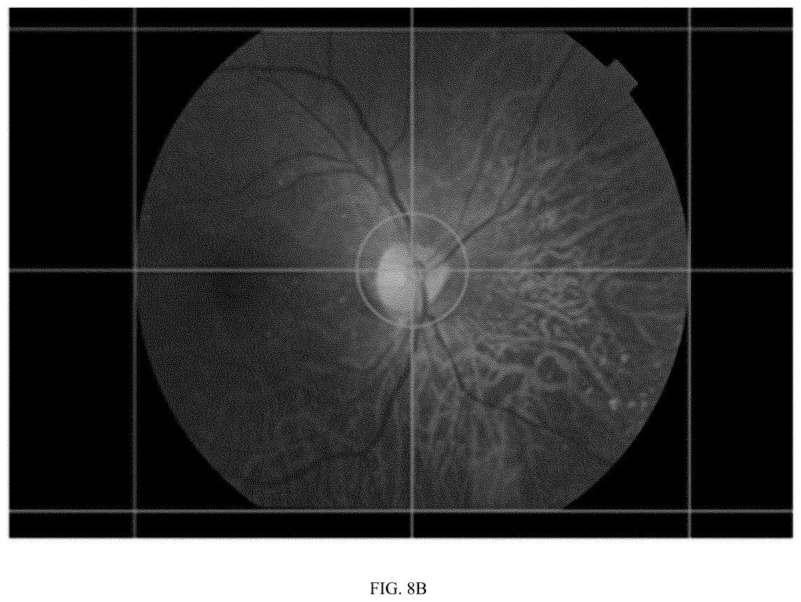

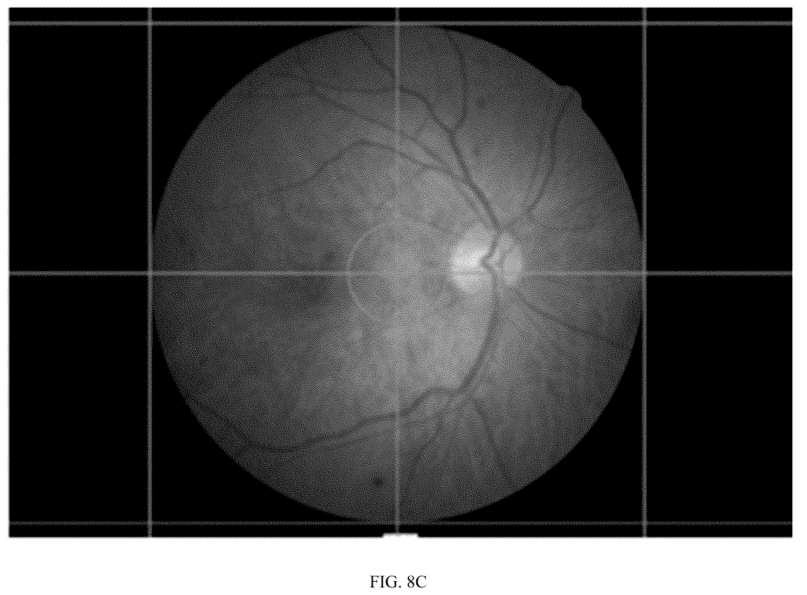

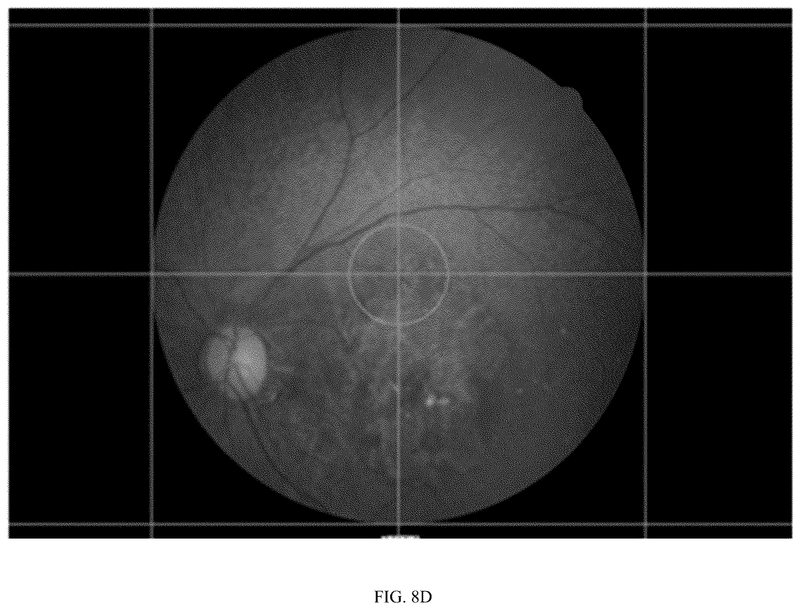

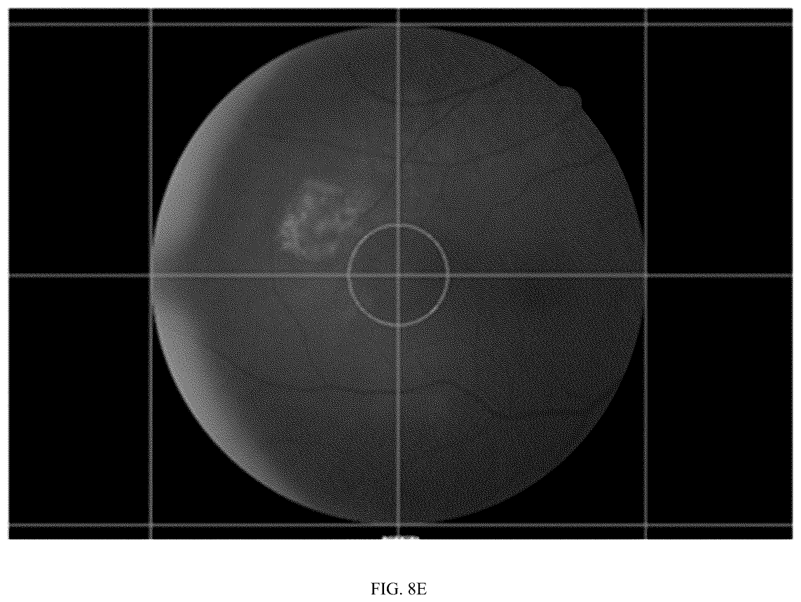

The inventors have tested the pre-diagnosis image assessment (published) and AD classification DL modules (non-published) in retrospective multi-center cohorts. The performance and heatmaps are shown in Table 1, Table 2, and FIGS. 4A-4D. The inventors have now developed an embodiment providing a cloud-based web infrastructure to integrate selected DL modules with an information management system for clinical deployment. FIGS. 5A-5C illustrate the operation of an embodiment for image uploading by end users and AI output of image assessment, AD classification and heatmap visualization. Embodiments are contemplated to integrate with one or more retinal photography devices and evaluate the performance of such photography devices prospectively.

TABLE 1
Performances of the pre-diagnosis image assessment module.

| | | | | |
|---|---|---|---|---|
| | AUROC | Sensitivity, % | Specificity, % | Accuracy, % |
| Dataset | (95% CI) | (95% CI) | (95% CI) | (95% CI) |

Image-quality

| | | | | |
|---|---|---|---|---|
| Internal | 0.975 | 92.1 | 98.3 | 92.5 |
| validation | (0.956-0.995) | (88.2-95.5) | (91.5-100) | (89.0-95.6) |
| External-1 | 0.999 | 99.3 | 100 | 99.3 |
| | (0.999-1.000) | (98.9-99.7) | (100-100) | (99.0-99.6) |
| External-2 | 0.987 | 95.0 | 96.4 | 95.1 |
| | (0.981-0.993) | (92.4-96.9) | (93.7-98.7) | (92.9-96.8) |

Field-of-view

| | | | | |
|---|---|---|---|---|
| Internal | 1.000 | 100 | 100 | 100 |
| validation | (1.000-1.000) | (100-100) | (100-100) | (100-100) |
| External-1 | 1.000 | 100 | 100 | 100 |
| | (1.000-1.000) | (100-100) | (100-100) | (100-100) |
| External-2 | 1.000 | 100 | 100 | 100 |
| | (1.000-1.000) | (99.8-100) | (99.9-100) | (99.9-100) |

Laterality-of-the-eye

| | | | | |
|---|---|---|---|---|
| Internal | 1.000 | 100 | 100 | 100 |
| validation | (1.000-1.000) | (100-100) | (100-100) | (100-100) |
| External-1 | 0.999 | 99.7 | 99.7 | 99.7 |
| | (0.998-1.000) | (99.4-100) | (99.4-100) | (99.5-99.9) |
| External-2 | 0.985 | 94.0 | 95.8 | 94.8 |
| | (0.982-0.989) | (91.7-96.2) | (93.3-97.7) | (94.1-95.6) |

AUROC = the area under the receiver operating characteristic curve,
CI = confidence interval

TABLE 2
The performance of the deep-learning models for Alzheimer's disease
classification in the internal validation and external testing datasets.

| | | | | | |
|---|---|---|---|---|---|
| Accuracy, | Sensitivity, | Specificity, | | | |
| % | % | % | AUROC | PPV, % | NPV, % |

Direction-1

| | | | | | | |
|---|---|---|---|---|---|---|
| Internal | 80.0 ± 2.8 | 75.1 ± 2.2 | 82.6 ± 4.4 | 0.84 ± 0.02 | 69.9 ± 6.9 | 86.1 ± 1.0 |
| validation | | | | | | |
| External-1 | 81.3 ± 9.3 | 73.8 ± 15.1 | 95.8 ± 9.3 | 0.81 ± 0.14 | 92.9 ± 15.8 | 66.3 ± 20.5 |
| External-2 | 89.3 ± 13.7 | 91.7 ± 16.7 | 90.0 ± 20.0 | 0.80 ± 0.24 | 87.6 ± 24.9 | 95.0 ± 10.0 |
| External-3 | 83.5 ± 17.8 | 87.8 ± 21.7 | 99.7 ± 0.7 | 0.79 ± 0.20 | 98.8 ± 2.6 | 82.8 ± 37.0 |
| External-4 | 79.5 ± 22.4 | 85.0 ± 22.4 | 90.0 ± 22.4 | 0.73 ± 0.29 | 85.0 ± 33.5 | 82.5 ± 27.4 |
| External-5 | 89.6 ± 4.2 | 100.0 ± 0.0 | 88.6 ± 4.7 | 0.89 ± 0.05 | 44.4 ± 9.6 | 100.0 ± 0.0 |

Direction-2

| | | | | | | |
|---|---|---|---|---|---|---|
| Internal | 77.0 ± 3.5 | 84.5 ± 5.6 | 73.0 ± 6.6 | 0.82 ± 0.03 | 62.9 ± 5.8 | 90.0 ± 2.8 |
| validation | | | | | | |
| External-1 | 79.1 ± 8.6 | 83.2 ± 10.1 | 80.3 ± 25.9 | 0.76 ± 0.17 | 85.4 ± 15.8 | 69.1 ± 22.7 |
| External-2 | 79.3 ± 7.4 | 91.8 ± 14.4 | 80.0 ± 27.4 | 0.63 ± 0.18 | 89.1 ± 17.4 | 89.3 ± 15.3 |
| External-3 | 88.9 ± 15.8 | 90.9 ± 17.4 | 99.7 ± 0.7 | 0.84 ± 0.21 | 98.9 ± 2.4 | 83.7 ± 35.6 |
| External-4 | 85.3 ± 10.7 | 80.0 ± 27.4 | 93.3 ± 14.9 | 0.72 ± 0.23 | 97.8 ± 5.0 | 86.7 ± 18.2 |
| External-5 | 96.2 ± 3.3 | 100.0 ± 0.0 | 95.9 ± 3.6 | 0.96 ± 0.04 | 72.2 ± 25.5 | 100.0 ± 0.0 |

Direction-3

| | | | | | | |
|---|---|---|---|---|---|---|
| Internal | 80.1 ± 2.2 | 81.2 ± 5.1 | 79.6 ± 4.4 | 0.88 ± 0.01 | 59.1 ± 4.3 | 92.4 ± 1.6 |
| validation | | | | | | |
| External-1 | 65.9 ± 5.9 | 58.9 ± 17.4 | 86.8 ± 19.7 | 0.64 ± 0.11 | 91.0 ± 14.6 | 52.1 ± 13.3 |
| External-2 | 80.3 ± 13.4 | 84.7 ± 13.9 | 82.5 ± 23.6 | 0.71 ± 0.14 | 77.8 ± 27.2 | 89.8 ± 7.4 |
| External-3 | 84.5 ± 9.4 | 88.3 ± 12.6 | 86.0 ± 15.7 | 0.79 ± 0.15 | 81.4 ± 18.5 | 86.4 ± 21.0 |
| External-4 | 79.9 ± 6.8 | 69.3 ± 21.3 | 92.5 ± 11.2 | 0.67 ± 0.05 | 92.7 ± 10.1 | 78.7 ± 13.3 |
| External-5 | 72.7 ± 10.3 | 100.0 ± 0.0 | 70.4 ± 10.7 | 0.76 ± 0.10 | 25.0 ± 11.7 | 100.0 ± 0.0 |

### EXAMPLE 2

Deep-Learning-Based Pre-Diagnosis Assessment Module for Retinal photographs: A Multicenter Study (Yuen V. et al., Transl Vis Sci Technol. 2021; 10(11):16. https://doi.org/10.1167/tvst.10.11.16, which is hereby incorporated by reference in its entirety, including any tables and figures.)

The subject disclosure in this application focuses on an AI-aided classification system for Alzheimer's disease (AD) screening from retinal photographs. In contrast, related art systems teach developing and validating a deep learning-based pre-diagnosis quality control method module for retinal photographs, targeting image quality, field of view, and laterality of the eye. An exemplary and non-limiting list of advantages of the subject invention over related art systems includes the following.

Embodiments of the subject invention provide a system for AD screening using retinal photographs, whereas related art teaches systems and methods for creating an AI-driven pre-diagnosis assessment module for general eye disease detection and screening from retinal photographs.

Related art teaches the development and validation of well-established CNN architecture (EfficientNet-B0 and MobileNetV2) for prediction. However, certain embodiments of the subject invention provide new techniques to integrate features from different retinal photographs, new networks with unsupervised domain adaptation technique to address dataset shifts between the different center data, and a deep learning-based AD classification module with additional heatmaps for visualization.

Embodiments of the subject invention provide three different deep learning models to predict AD-dementia/non-demented probabilities from three directions: both eyes' four image (Direction-1, or bilateral), both eyes' four images combining demographical information (Direction-2, or hybrid), and single eye's two images (Direction-3, or unilateral).

Embodiments of the subject invention provide a complimentary risk profiling tool for AD to assist physicians in identifying asymptomatic individuals with a higher likelihood of having AD in the community.

Embodiments of the subject invention provide systems and methods to specifically target AD screening, offering numerous advantages over the related art's more general application for eye disease detection and screening.

### EXAMPLE 3

A deep learning model for detection of Alzheimer's disease based on retinal photographs: a retrospective, multicenter case-control study (www.thelancet.com/digital-health Published online Sep. 30, 2022. https://doi.org/10.1016/S2589-7500(22)00169-8 1) which is hereby incorporated by reference in its entirety, including any tables and figures.).

There is no simple model in related art to screen for Alzheimer's disease, partly because the diagnosis of Alzheimer's disease itself is complex—typically involving expensive and sometimes invasive tests not commonly available outside highly specialized clinical settings. The inventors aimed to develop a deep learning algorithm that could use retinal photographs alone, which is the most common method of non-invasive imaging the retina to detect Alzheimer's disease-dementia.

In this retrospective, multicenter case-control study, the inventors trained, validated, and tested a deep learning algorithm to detect Alzheimer's disease-dementia from retinal photographs using retrospectively collected data from 11 studies that recruited patients with Alzheimer's disease-dementia and people without disease from different countries. The main aim was to develop a bilateral model to detect Alzheimer's disease-dementia from retinal photographs alone. The inventors designed and internally validated the bilateral deep learning model using retinal photographs from six studies. The inventors used the EfficientNet-b2 network as the backbone of the model to extract features from the images. Integrated features from four retinal photographs (optic nerve head-centered and macula-centered fields from both eyes) for each individual were used to develop supervised deep learning models and equip the network with unsupervised domain adaptation technique, to address dataset discrepancy between the different studies. The inventors tested the trained model using five other studies, three of which used PET as a biomarker of significant amyloid β burden (testing the deep learning model between amyloid β positive vs amyloid β negative).

A total of 12,949 retinal photographs from 648 patients with Alzheimer's disease and 3240 people without the disease were used to train, validate, and test the deep learning model. In the internal validation dataset, the deep learning model had 83.6% (SD 2.5) accuracy, 93.2% (SD 2.2) sensitivity, 82.0% (SD 3.1) specificity, and an area under the receiver operating characteristic curve (AUROC) of 0.93 (0.01) for detecting Alzheimer's disease-dementia. In the testing datasets, the bilateral deep learning model had accuracies ranging from 79.6% (SD 15.5) to 92.1% (11.4) and AUROCs ranging from 0.73 (SD 0.24) to 0.91 (0.10). In the datasets with data on PET, the model was able to differentiate between participants who were amyloid β positive and those who were amyloid β negative: accuracies ranged from 80.6 (SD 13.4%) to 89.3 (13.7%) and AUROC ranged from 0.68 (SD 0.24) to 0.86 (0.16). In subgroup analyses, the discriminative performance of the model was improved in patients with eye disease (accuracy 89.6% [SD 12.5%]) versus those without eye disease (71.7% [11.6%]) and patients with diabetes (81.9% [SD 20.3%]) versus those without the disease (72.4% [11.7%]).

The above results show retinal photograph-based deep learning algorithm according to an embodiment of the subject invention can detect Alzheimer's disease with good accuracy, showing its ability for screening Alzheimer's disease in a community setting.

Alzheimer's disease, the most common form of dementia, is a global public health problem.1 Diagnosis of Alzheimer's disease is complex and typically involves expensive and sometimes invasive tests not commonly available outside of highly specialized clinical settings. For example, biomarkers of amyloid β and phosphorylated tau measured through cerebrospinal fluid assessments,

PET scans, and plasma assays are helpful for Alzheimer's disease diagnosis, but these tests are not suitable for screening possible Alzheimer's disease in primary care or community settings.² Of note, because Alzheimer's disease treatment is available,³ simple, accessible, and sensitive community-based screening tests would substantially improve population-based strategies to manage Alzheimer's disease.

The retina, a neurosensory layered tissue lining the back of the eye and directly connected to the brain via the optic nerve, has long been considered a platform to study disorders in the CNS because it is an accessible extension of the brain in terms of embryology, anatomy, and physiology.^{4,5} Retinal changes in Alzheimer's disease have been shown in post-mortem histopathological studies.^{6,7} This concept is supported by clinical studies showing a range of retinal changes in patients with Alzheimer's disease, such as changes in the retinal vasculature (e.g., vessel caliber and retinopathy signs), the optic nerve, and the retinal nerve fiber layer.^{5,8} These features can be non-invasively imaged using digital retinal photography, which is now widely available at a low cost in primary care optometry and community settings.

Artificial intelligence (AI), particularly deep learning, allows algorithms to extract both known and unknown features from images for accurate detection of a condition, without the need for manual identification of specific features. Deep learning has been applied to retinal photographs for detecting various ophthalmic diseases (such as diabetic retinopathy,⁹ optic disc papilledema,¹⁰ glaucoma,¹¹ and age-related macular degeneration¹²). Furthermore, deep learning approaches can also detect systemic diseases based on retinal photographs (e.g., systemic biomarkers,¹³ cardiovascular disease,¹⁴ diabetes,¹⁵ chronic kidney disease,¹⁶ and hepatobiliary diseases,¹⁷). However, the role of deep learning approaches in detecting Alzheimer's disease from retinal photographs has yet to be determined in related art systems, and has only now been shown in embodiments of the subject invention.

Embodiments of the subject invention provide a novel deep learning algorithm for automated detection of Alzheimer's disease-dementia from retinal photographs alone to determine its possible use for Alzheimer's disease screening. To address this, the inventors trained, validated, and tested the deep learning models using retinal photographs from 11 clinical studies. The inventors also tested the ability of our deep learning model to differentiate patients who were amyloid β positive from those who were amyloid β negative.

In this retrospective, multicenter case-control study, the inventors trained, validated, and tested a deep learning model for detecting Alzheimer's disease from retrospectively collected retinal photographs from 648 patients with Alzheimer's disease and 3240 patients who did not have the disease. This included 11 clinical studies done at eight centers in four countries (Hong Kong Special Administrative Region, China, Singapore, the UK, and the USA; see Example 4). The inclusion and exclusion criteria for patients in each of the 11 studies are reported in Example 4. For all participants, four retinal photographs (optic nerve head-centered and macula-centered images from both eyes) were used for the model development.

This multicenter study was approved by the human ethics boards of the Joint Chinese University of Hong Kong-New Territories East Cluster Clinical Research Ethics Committee, Hong Kong Special Administrative Region, China, and local research ethics committees in each center. The 11 studies used to generate the test populations were all done according to the Declaration of Helsinki, with written informed consent obtained from each participant or their guardians. The STARD guideline was used for reporting in the current study.

The main aim of this study was to develop a bilateral deep learning model that outputted participant-level detection results (i.e., Alzheimer's disease-dementia or no dementia) accounting for Alzheimer's disease features from optic nerve head-centered and macula-centered images from both eyes. The inventors used retinal photographs from six studies with labels of either Alzheimer's disease-dementia or no dementia as primary datasets (i.e., source domain; primary 1-6; Example 4) for the development and internal validation of the deep learning model. The inventors tested the trained deep learning models with five non-overlapping studies that had labels of Alzheimer's disease-dementia or no dementia (i.e., e, target domain; testing datasets 1-5; Example 4). The image quality was labelled by three trained human graders (ARR, VTTC, and KS). Only gradable retinal photographs were used. If more than 25% of the peripheral area of the retina was unobservable due to artifacts, including the presence of foreign objects, out-of-focus imaging, blurring, and extreme illumination conditions and if the center region of the retina had significant artifacts that would affect analysis, the photograph was considered ungradable. The inter-grader reliability was high, with Cohen's K coefficients ranging from 0.868 to 0.925. If grader 2 (VTTC) and grader 3 (KS) could not make a decision as to whether an image should be included (e.g., retinal photographs with borderline quality), the senior grader (grader 1 [ARR]) made final decisions.¹⁸ The labelling of Alzheimer's disease-dementia in all studies followed the Diagnostic and Statistical Manual of Mental Disorders, 4th edition, criteria for dementia syndrome (Alzheimer's type) and National Institute of Neurological and Communicative Disorders and Stroke and the Alzheimer's Disease and Related Disorders Association criteria for probable or possible Alzheimer's disease. Retinal photographs were labelled as no dementia when the participant had no objective cognitive impairment evident in the neuropsychological assessments and no history of neurodegenerative diseases.

Three testing sets (testing set 1-3; Example 4) also included data from amyloid-PET scan examinations following intravenous 11C-Pittsburgh compound B to quantify amyloid β deposition from a series of brain regions. The retinal photographs with amyloid-PET scan available were additionally labelled as either amyloid β positive or amyloid β negative based solely on the standardized uptake value ratio with reference to the locally validated cutoff value, regardless of their clinical diagnosis. The details of the primary and testing datasets are described in Example 4.

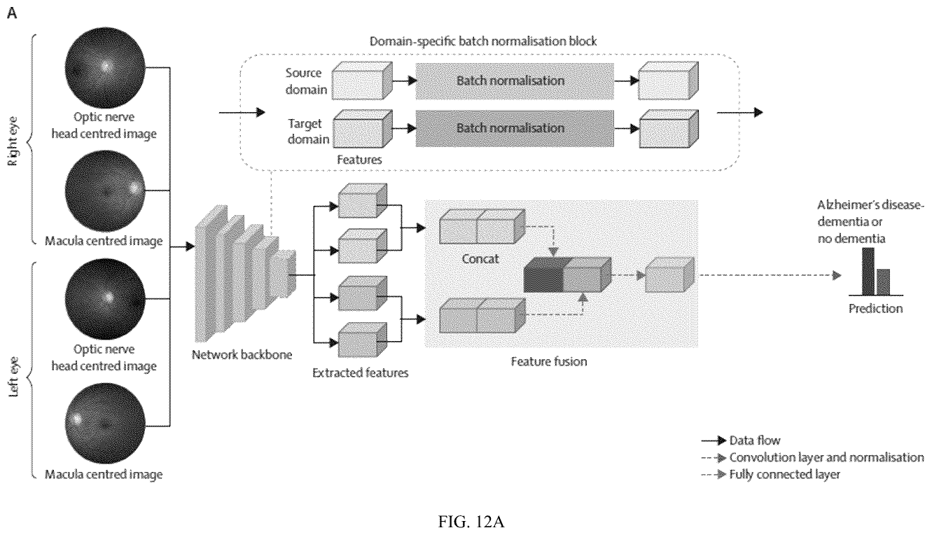

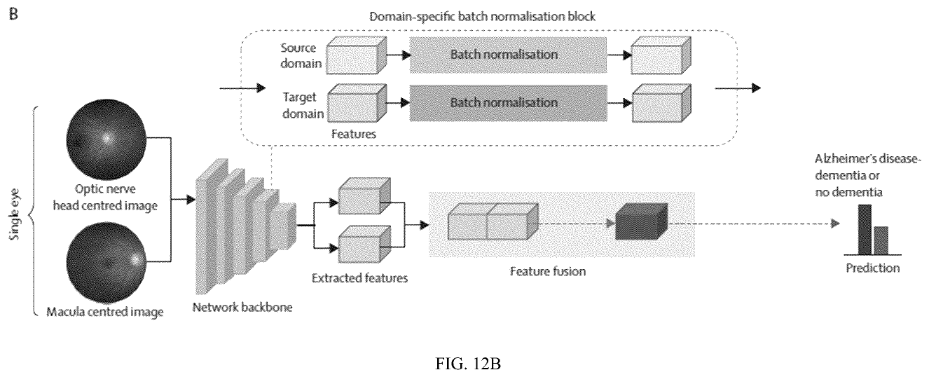

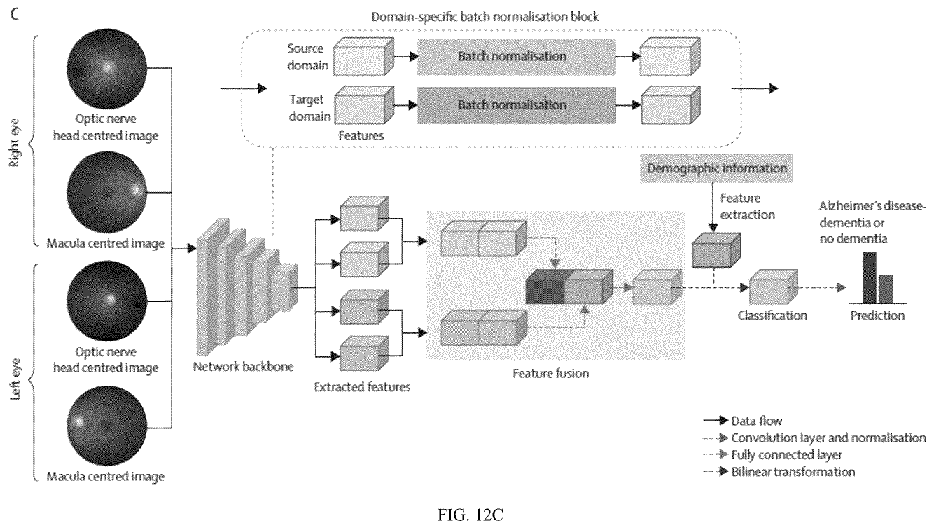

Because the labelling input and classification output were dependent on the individual participant rather than the image, the deep learning model was designed to integrate features of Alzheimer's disease from four retinal photographs from each participant (i.e., both optic nerve head-centered and macula-centered fields from both eyes). The datasets were split at a participant level to inhibit or avoid information leakage and performance overestimation. The method consisted of four phases. In the first phase, the inventors designed a basic model, using EfficientNet-b2,¹⁹ as the backbone for feature extractor, which is based on only one single retinal photograph for the detection of Alzheimer's disease-dementia (Example 4). The inventors then proposed a bilateral model based on four retinal photographs, which learned Alzheimer's disease-related features from optic nerve head-centered and macula-centered retinal photographs from both eyes (FIG. 12A). Specifically, the inventors designed an adaptative feature fusion technique to integrate the extracted information from multiple retinal photographs from both eyes. The bilateral model (main aim of this study) outputted participant-level detection results (i.e., Alzheimer's disease-dementia or no dementia) accounting for Alzheimer's disease features from both eyes. If multiple paired images were available for the same individual, the inventors sorted the predictive values for all the paired images and used the image with the median value for the final participant-level prediction. In the second phase, the inventors developed a unilateral model for single eye analysis because individuals might have an ungradable retinal photograph from one eye (e.g., due to severe cataract; FIG. 12B). In the third phase, the inventors trained a hybrid model that could consider risk factors of Alzheimer's disease (i.e., age, gender, and presence or absence of hypertension and diabetes; FIG. 12C). Finally, the inventors trained a “risk factors alone” model for Alzheimer's disease prediction (Example 4) for comparison with the bilateral model.

The inventors used unsupervised domain adaptation with domain-specific batch normalization to address data heterogeneity and domain shift problems and to improve the model performance. Unsupervised domain adaptation is a type of learning framework that can transfer knowledge learned from a larger number of annotated training data in the source domains to target domains with unlabeled data only. Domain-specific batch normalization is a building block for deep neural networks for which the source domain and the target domain datasets have their own separate batch normalization layer for training and extraction of hyper-parameters. This design addressed characteristics specific to each domain that are not compatible within a single model while retaining domain-invariant information that is common to all domains. In brief, the labelled source domain dataset was first used for training in a supervised way to generate an unsupervised domain adaption network. This unsupervised domain adaptation network was then used to generate pseudo-labels for unlabeled data in the target domain. The final classification network was subsequently trained with full supervision using labelled data from the source domain and pseudo-labelled data from the target domains. Through the fusion of the domain-independent and domain-dependent knowledge learning, the deep learning models could transfer discriminative features from the labelled source domain to the unlabeled target domain (i.e., domain adaptation) and improve the classification performance on the target domain. Due to poor model transfer capability of the domain adaptation-based method, the inventors trained one model for each testing dataset to obtain the information, such as image style distribution of unlabeled testing datasets.

Furthermore, to better understand discriminative features between patients with Alzheimer's disease-dementia and participants without the disease, the inventors used Gradient-weighted Class Activation Mapping (i.e., heatmap) to visualize the features extracted from the last convolutional layer. Details of the network architecture, training details, and objective functions were described in Example 4.

The inventors used the testing datasets to evaluate the model performance at a participant level on three aspects: clinically diagnosed Alzheimer's disease-dementia versus no dementia, individuals who were amyloid β positive versus individuals who were amyloid β negative, and individuals who had clinically diagnosed Alzheimer's disease-dementia and were amyloid β positive versus those who had no cognitive impairment and were amyloid β negative. Models were evaluated based on the following metrics from the five-fold cross validation: the area under the receiver operating characteristic curve (AUROC) and values for accuracy, sensitivity, and specificity for which the cutoff point was the largest Youden Index in each dataset.

In subgroup analyses, the inventors combined the testing 1-3 datasets and stratified individuals on the basis of the presence of eye disease from retinal photographs and diabetes diagnosis status to evaluate discriminative performance. The performance of the unilateral model was also compared between right eyes and left eyes.

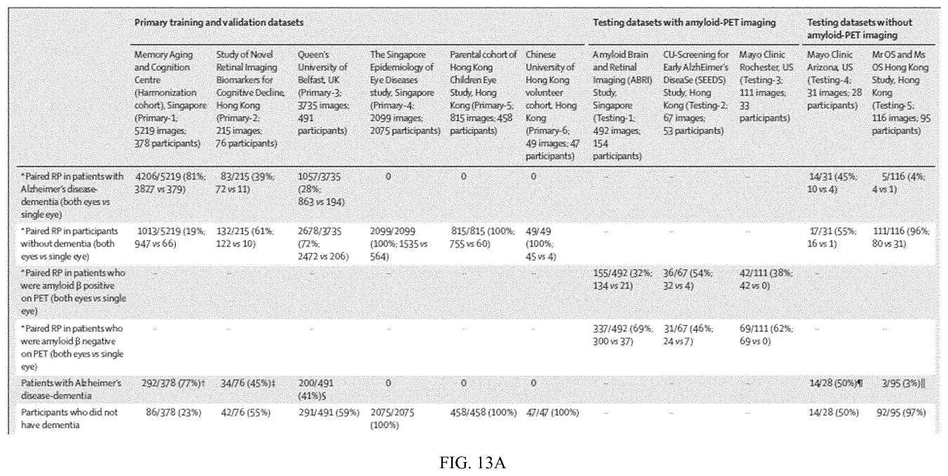

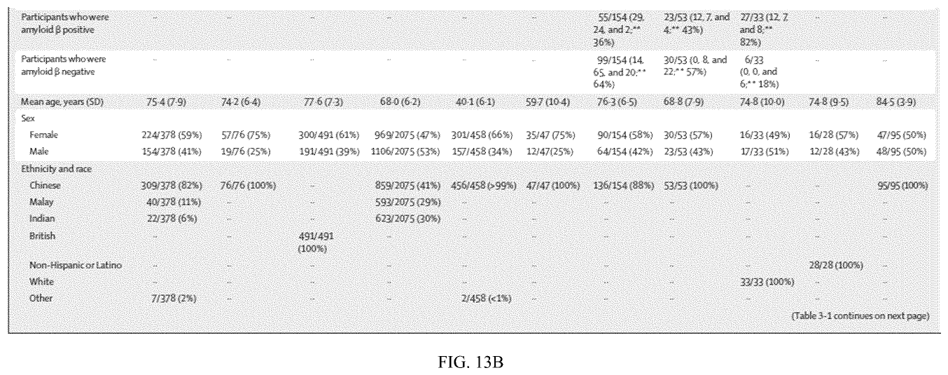

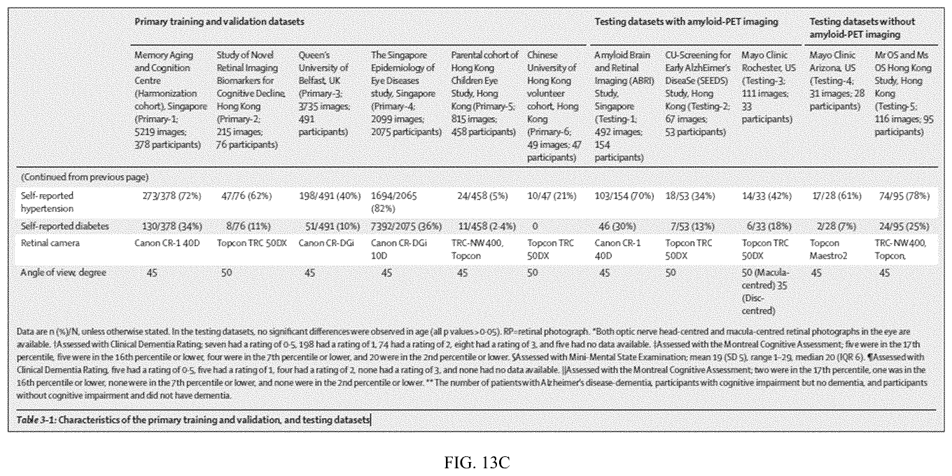

A total of 5598 retinal photographs from 648 individuals with Alzheimer's disease and 7351 retinal photographs from 3240 people without the disease were used to train, validate, and test the deep learning models. The characteristics of the primary training, internal validation, and testing datasets at a participant level are reported in FIGS. 13A-13C (“Table 3-1” from Example 3.)

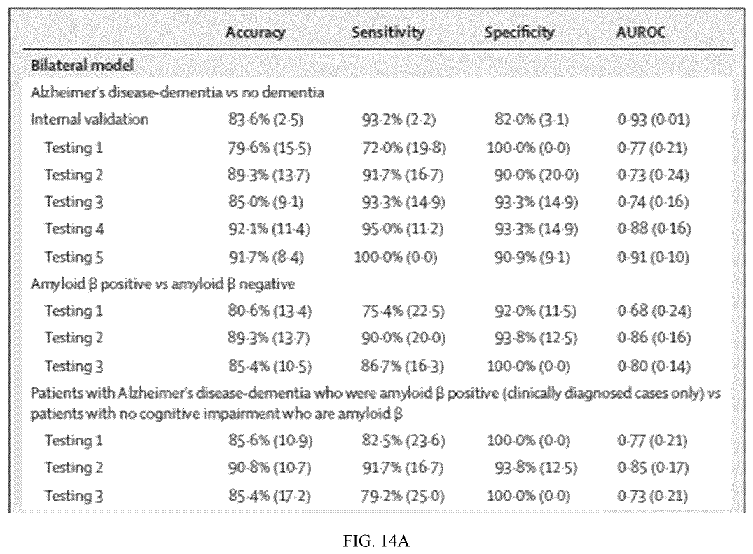

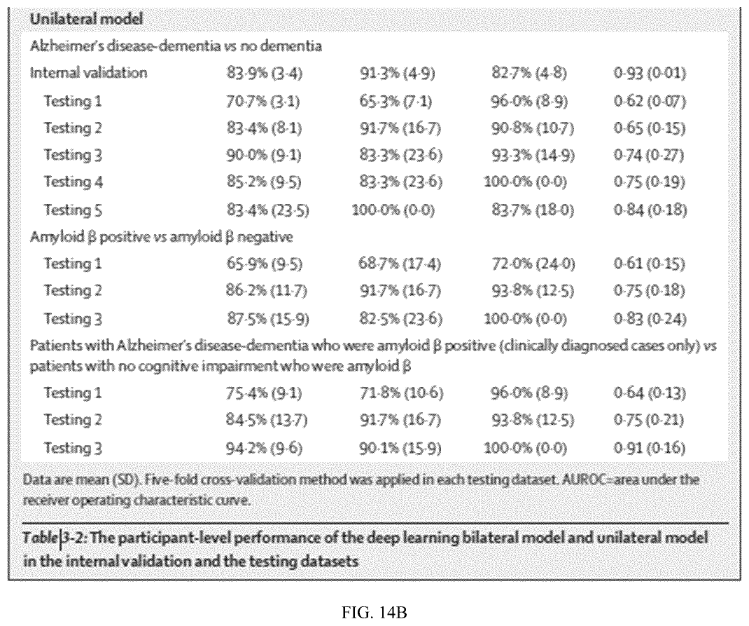

In the internal validation dataset, the bilateral model had 83.6% (SD 2.5) accuracy, 93.2% (SD 2.2) sensitivity, 82.0% (SD 3.1) specificity, and an AUROC of 0.93 (0.01) for detection of Alzheimer's disease-dementia. For differentiation between patients with Alzheimer's disease-dementia and participants who did not have the disease, both bilateral and unilateral models had accuracies of more than 83% and AUROCs of more than 0.9 in internal validation (FIGS. 14A-14B, “Table 3-2”). During testing, the bilateral model had accuracies ranging from 79.6% (SD 15.5) to 92.1% (11.4) and AUROCs ranging from 0.73 (SD 0.24) to 0.91 (0.10; FIGS. 14A-14B, “Table 3-2”). In the three testing datasets with known amyloid β status from PET, the bilateral model was able to differentiate between those who were amyloid β positive and those who were amyloid β negative with an accuracy ranging from 80.6% (SD 13.4) to 89.3% (13.7), and an AUROC ranging from 0.68 (SD 0.24) to 0.86 (0.16; FIGS. 14A-14B, “Table 3-2”), which was similar to the model's ability to differentiate between people who were clinically diagnosed with Alzheimer's disease and were amyloid β positive from people without the disease and were amyloid β negative. Heatmaps differentiating true-positive and true-negative examples are reported in Example 4. The performance of the bilateral model was better than that of the unilateral model in the testing (FIGS. 14A-14B, “Table 3-2”). The performance of the unilateral model was largely similar between right eyes and left eyes, Example 4.

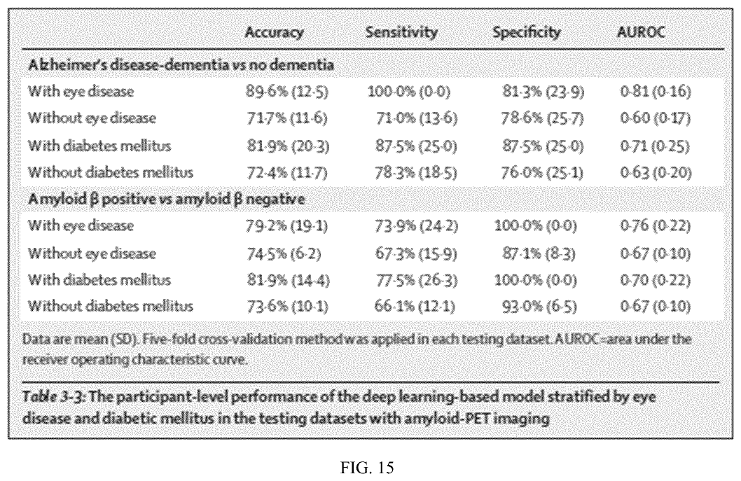

In the subgroup analysis, the ability of the model to differentiate between people with Alzheimer's disease-dementia and those without the disease and those who were amyloid β positive from those who were amyloid β negative was improved in patients with concomitant eye disease (accuracy 89.6% [SD 12.5%]) versus those without eye disease (71.7% [11.6%]; FIG. 15, “Table 3-3”) and patients with diabetes (81.9% [SD 20.3%]) versus those without diabetes (72.4% [11.7%]). Of note, the model performance was maintained when risk factors of Alzheimer's disease (i.e., age, gender, and presence of hypertension and diabetes) were included in the model (hybrid model; Example 4). Except for the testing set 5, which had a similar performance, the bilateral model had higher accuracy than the risk factors alone model (Example 4).

Compared with the Hong Kong version of the Montreal Cognitive Assessment for Alzheimer's disease-dementia detection in a community-based cohort, our bilateral model's assessment of testing set 5 had higher sensitivity (100% vs 50%) and a higher AUROC (0.91 vs 0.75; Example 4).

Unsupervised domain adaptation with domain-specific batch normalization was used in the testing datasets to address the issue of data heterogeneity and domain shift problems. After domain adaptation, the model performance was generally improved, suggesting that the model also learned discriminative features from the source domain for Alzheimer's disease detection (Example 4).

In this study, the inventors developed, validated, and tested a novel, retinal photograph-based deep learning algorithm according to an embodiment of the subject invention to detect individuals with Alzheimer's disease, using an unsupervised domain adaptation deep learning technique to improve its general usability. The provided deep learning algorithm showed consistently accurate performance for differentiating between patients with Alzheimer's disease-dementia and individuals with no dementia. In particular, the performance was similar for differentiating between people who were amyloid β positive from those who were amyloid β negative. In addition, the provided deep learning algorithm had good performance in the presence of concomitant eye diseases (e.g., age-related macular degeneration), thus allowing screening in optometry and ophthalmology settings.

Embodiments of the subject invention provide the first deep learning model to detect Alzheimer's disease from retinal photographs alone. In related art, Wisely and colleagues²⁰ proposed a deep learning system to predict Alzheimer's disease using images and measurements from multiple ocular imaging modalities (e.g., optical coherence tomography, optical coherence tomography angiography, ultra-widefield retinal photography, and retinal auto-fluorescence) and patient data. By contrast, embodiments of the subject invention predict Alzheimer's disease based on retinal photographs only, thus improving the efficiency and potential cost-effectiveness of the algorithm. The provided algorithm advantageously employs two advanced deep learning techniques: unsupervised domain adaptation and feature fusion. The novel application of these two techniques addresses two significant challenges: (1) data distribution discrepancy between training and validation and testing datasets, and (2) the integration from multiple optic nerve head-centered and macula-centered retinal photographs from both eyes. With this deep learning architecture, embodiments are transferrable to a new center without developing a new deep learning model. Retrospective data can be collected from this specific center for unsupervised domain adaptation, and the model can subsequently be refined to keep the deep learning model up to date.

To increase applicability, the inventors intentionally included retinal photographs with concomitant eye disease in the training dataset because age-associated eye conditions (e.g., age-related macular degeneration and glaucoma) are common in people older than 60 years. Excluding eyes with these conditions might also introduce selection bias because studies have shown the patients with Alzheimer's disease are more likely to have age-associated macular degeneration and glaucoma.^{5,21,22} The provided deep learning algorithm retained a robust ability to differentiate between people who had and did not have Alzheimer's disease, even in the presence of concomitant eye diseases. These findings suggest that Alzheimer's disease has unique retinal features that are distinguishable from other eye diseases. Furthermore, patients with type 2 diabetes are at higher risk of cognitive impairment.23 Embodiments of the provided deep learning algorithm performed well without significant interference from concomitant diabetes, and while not being bound by theory, the inventors hypothesize this can be attributed to its similarity with deep learning-based diabetic retinopathy screening.²⁴ However, the performance of the model in participants without eye disease dropped. While not being bound by theory, the inventors hypothesize that an overlap in pathophysiological features shared between Alzheimer's disease and eye diseases might enhance the identification of Alzheimer's disease-associated features from retinal imaging.

The inventors developed a supplementary unilateral model, which can estimate the risk of Alzheimer's disease based on retinal photographs from a single eye. A unilateral model is essential for community screening of Alzheimer's disease because retinal photograph of one eye might not be assessable due to media opacity (e.g., cataract). Results suggest that the unilateral model can also reliably predict Alzheimer's disease-dementia based on unilateral retinal photographs.

The provided retinal photograph-based deep learning model addresses a current gap in Alzheimer's disease screening, in which under-diagnosis of dementia is highly prevalent.²⁵ Early diagnosis of Alzheimer's disease relies on a complex series of cognitive tests, clinical assessments, supportive evidence from neuroimaging (e.g., PET), and cerebrospinal fluid biomarker evidence, with the definitive diagnosis only confirmed post mortem.²⁶ Therefore, patients with Alzheimer's disease are usually diagnosed late after the onset of debilitating dementia when there has already been extensive brain neurodegeneration that might not be amenable to any disease-modifying treatment.²⁷ Embodiments of the retinal photograph-based deep learning model provide a simple, low-cost, low labor-dependent approach to identify potential Alzheimer's disease-dementia patients in community settings with reasonable accuracy and sensitivity. In certain embodiments the identified patients can then be referred to and followed up at tertiary facilities with diagnostic evaluation and subsequent multidisciplinary managements. The detection of Alzheimer's disease based on retinal photographs can also leverage existing community eye-care infrastructure (e.g., optometry or primary care networks) that enables opportunistic Alzheimer's disease screening during routine screening for common eye diseases, such as diabetic retinopathy and glaucoma. With advances in telemedicine and the increasing popularity of non-mydriatic digital retinal cameras and smartphone-based cameras, access to retinal photography is expected to increase. Because retinal photograph-based deep learning approaches could be used for screening Alzheimer's disease-dementia, it is contemplated within the scope of certain embodiments of the subject invention to improve sensitivity and specificity when combining retinal photography with blood-based biomarkers, which have been shown to correlate with brain amyloid and tau burden—the upstream pathology of Alzheimer's disease. In addition, identifying prodromal and preclinical Alzheimer's disease and predicting progression to dementia in those with mild cognitive impairment is contemplated. Advances in retinal photograph-based deep learning model development in this direction are also contemplated for future institutional and clinical applications.

Embodiments have been proven effective across a diverse clinical sample, with datasets from multiethnic, multicountry cohorts and in different clinical settings. Embodiments have been validated in five testing datasets, three of which included amyloid-PET scan. Furthermore, the inventors used unsupervised domain adaptation with domain-specific batch normalization to address data discrepancy from different datasets, which largely improved the proposed model's generalizability and its potential feasibility in other unseen clinical settings. It is further contemplated within the scope of the subject invention that after integration with prediagnosis assessment deep learning models,¹⁸ certain embodiments can provide an integrated and comprehensive deep learning pipeline for Alzheimer's disease screening in the community.

Pathological studies suggest that clinical Alzheimer's disease diagnostic sensitivity ranges between 70.9% and 87.3%, and specificity between 44.3% and 70.8%.²⁹ Because the labelling of training datasets are often based on clinician-derived diagnosis, the development of any deep learning algorithm can include retinal photographs from individuals incorrectly labelled as having Alzheimer's disease. Embodiments provide training and/or testing in datasets with PET imaging to mitigate this concern.

Embodiments provide a validated and tested retinal photograph-based deep learning system and method for detecting and treating Alzheimer's disease-dementia, advantageously including a unique and generalizable model useful in community and public health settings to screen for and better treat Alzheimer's disease.

### REFERENCES FOR EXAMPLE 3: MULTICENTER CASE-CONTROL STUDY

1 No authors listed. 2021 Alzheimer's disease facts and figures. Alzheimers Dement 2021; 17: 327-406.
2 Olsson B, Lautner R, Andreasson U, et al. CSF and blood biomarkers for the diagnosis of Alzheimer's disease: a systematic review and meta-analysis. Lancet Neurol 2016; 15: 673-84.
3 Alexander G C, Emerson S, Kesselheim A S. Evaluation of aducanumab for Alzheimer disease: scientific evidence and regulatory review involving efficacy, safety, and futility. JAMA 2021; 325: 1717-18.
4 London A, Benhar I, Schwartz M. The retina as a window to the brain-from eye research to CNS disorders. Nat Rev Neurol 2013; 9: 44-53.
5 Cheung C Y, Mok V, Foster P J, Trucco E, Chen C, Wong T Y. Retinal imaging in Alzheimer's disease. J Neurol Neurosurg Psychiatry 2021; 92: 983-94.
6 La Morgia C, Ross-Cisneros F N, Koronyo Y, et al. Melanopsin retinal ganglion cell loss in Alzheimer disease. Ann Neurol 2016; 79: 90-109.
7 Hinton D R, Sadun A A, Blanks J C, Miller C A. Optic-nerve degeneration in Alzheimer's disease. N Engl J Med 1986; 315: 485-87.
8 Lee C S, Larson E B, Gibbons L E, et al. Associations between recent and established ophthalmic conditions and risk of Alzheimer's disease. Alzheimers Dement 2019; 15: 34-41.
9 Ting D S W, Cheung C Y, Lim G, et al. Development and validation of a deep learning system for diabetic retinopathy and related eye diseases using retinal images from multiethnic populations with diabetes. JAMA 2017; 318: 2211-23.
10 Milea D, Najjar R P, Zhubo J, et al. Artificial intelligence to detect papilledema from ocular fundus photographs. N Engl J Med 2020; 382: 1687-95.
11 Liu H, Li L, Wormstone I M, et al. Development and validation of a deep learning system to detect glaucomatous optic neuropathy using fundus photographs. JAMA Ophthalmol 2019; 137: 1353-60.
12 Burlina P M, Joshi N, Pekala M, Pacheco K D, Freund D E, Bressler N M. Automated grading of age-related macular degeneration from color fundus images using deep convolutional neural networks. JAMA Ophthalmol 2017; 135: 1170-76.
13 Rim T H, Lee G. Kim Y, et al. Prediction of systemic biomarkers from retinal photographs: development and validation of deep-learning algorithms. Lancet Digit Health 2020; 2: e526-36.
14 Cheung C Y, Xu D, Cheng C Y, et al. A deep-learning system for the assessment of cardiovascular disease risk via the measurement of retinal-vessel calibre. Nat Biomed Eng 2021; 5: 498-508.
15 Zhang K, Liu X, Xu J, et al. Deep-learning models for the detection and incidence prediction of chronic kidney disease and type 2 diabetes from retinal fundus images. Nat Biomed Eng 2021; 5: 533-45.
16 Sabanayagam C, Xu D, Ting D S W, et al. A deep learning algorithm to detect chronic kidney disease from retinal photographs in community-based populations. Lancet Digit Health 2020; 2: e295-302.
17 Xiao W, Huang X, Wang J H, et al. Screening and identifying hepatobiliary diseases through deep learning using ocular images: a prospective, multicentre study. Lancet Digit Health 2021; 3: e88-97.
18 Yuen V, Ran A, Shi J, et al. Deep-learning-based pre-diagnosis assessment module for retinal photographs: a multicenter study. Transl Vis Sci Technol 2021; 10: 16.
19 Tan M, Le Q. EfficientNet: rethinking model scaling for convolutional neural networks. Proceedings of the 36th International Conference on Machine Learning 2019; 97: 6105-14.
20 Wisely C E, Wang D, Henao R, et al. Convolutional neural network to identify symptomatic Alzheimer's disease using multimodal retinal imaging. Br J Ophthalmol 2022; 106: 388-95.
21 Lee C S, Larson E B, Gibbons L E, et al. Associations between recent and established ophthalmic conditions and risk of Alzheimer's disease. Alzheimers Dement 2019; 15: 34-41.
22 Ohno-Matsui K. Parallel findings in age-related macular degeneration and Alzheimer's disease. Prog Retin Eye Res 2011; 30: 217-38.
23 Simó R, Ciudin A, Simó-Servat O, Hernández C. Cognitive impairment and dementia: a new emerging complication of type 2 diabetes—the diabetologist's perspective. Acta Diabetol 2017; 54: 417-24.
24 Xie Y, Nguyen Q D, Hamzah H, et al. Artificial intelligence for teleophthalmology-based diabetic retinopathy screening in a national programme: an economic analysis modelling study. Lancet Digit Health 2020; 2: e240-49.
25 Savva G M, Arthur A. Who has undiagnosed dementia? A cross-sectional analysis of participants of the aging, demographics and memory study. Age Ageing 2015; 44: 642-47.
26 Arvanitakis Z, Shah R C, Bennett D A. Diagnosis and management of dementia: review. JAMA 2019; 322: 1589-99.
27 Cummings J L, Morstorf T, Zhong K. Alzheimer's disease drug-development pipeline: few candidates, frequent failures. Alzheimers Res Ther 2014; 6: 37.
28 Wagner S K, Hughes F, Cortina-Borja M, et al. AlzEye: longitudinal record-level linkage of ophthalmic imaging and hospital admissions of 353 157 patients in London, UK. BMJ Open 2022; 12: e058552.
29 Beach T G, Monsell S E, Phillips L E, Kukull W. Accuracy of the clinical diagnosis of Alzheimer disease at National Institute on Aging Alzheimer Disease Centers, 2005-2010. J Neuropathol Exp Neurol 2012; 71: 266-73.

### EXAMPLE 4

### Supplementary Appendix to Case Study

The inventors used retinal photographs from 6 studies dated from 9th November 2003 to 29th September 2019 with labels of “Alzheimer's disease-dementia” and “no dementia” as primary training and validation datasets for model development. Potential for a data leak is remediated by data splitting on the subject level. For example, if one subject is grouped in the training set, all visits for that subject would only be used for training. There is no data cross between training and testing sets. These primary datasets include the following:
Primary-1: The Harmonization Cohort Study, Singapore
Primary-2: Study of Novel Retinal Imaging Biomarkers for Cognitive Decline, Hong Kong
Primary-3: The Belfast study, UK
Primary-4: The Singapore Epidemiology of Eye Diseases Study (SEED), Singapore
Primary-5: The parental cohort of Hong Kong Children Eye Study, Hong Kong, China
Primary-6: The CUHK volunteer cohort, Hong Kong, China

Primary-1: The Harmonization Cohort Study was a prospective memory clinic-based study.^{1,2} Participants with subjective complaints of memory problems and/or demonstrated cognitive impairment on neuropsychological assessment were recruited. Subjects with no dementia were recruited from both memory clinics and the community. All subjects were administered the clinical dementia rating (CDR) scale questionnaire, locally modified versions of the Mini Mental Status Examination (MMSE) and Montreal Cognitive Assessment (MoCA), and a standard neuropsychological battery locally validated for older Singaporeans by trained psychologists.

Primary-2: The study of Novel Retinal Imaging Biomarkers for Cognitive Decline was a prospective observational study to use retinal imaging as a novel biomarker for prognostic outcome measures of cognitive decline. Patients with Alzheimer's disease-dementia were recruited from the Cognitive Disorder Clinic or Memory Clinic of the Prince of Wales Hospital, and subjects with no dementia were recruited from an on-going community-based study in Hong Kong.

Primary-3: The Belfast study was a case-control study wherein Alzheimer's disease-dementia cases were recruited by an opportunistic strategy from the Royal Victoria Hospital, Belfast, UK.³ Subjects with no dementia were recruited from several sources: those responding to a press release on the study, friends of involved controls, carers of patients attending out-patient clinics, and patient-support groups.

Primary-4: The SEED study was a population-based study comprising adults residing in Singapore aged 40 to 80 years at baseline, from 3 major ethnic groups: Chinese, Indians, and Malays. Participants aged 60 years and older were administered the Abbreviated Mental Test (AMT) in SEED to assess cognitive function. Subjects with no dementia (i.e., screening-negative) were defined as a score >6/10 for participants with 0-6 years of formal education and >8/10 for those with more than 6 years of formal education.^{4,5}

Primary-5: The Hong Kong Children Eye Study was a population-based cohort study of eye conditions in children of Grade 1 to Grade 3 from primary schools in Hong Kong. Eye examination and retinal photography were also performed in the parents of the study subjects.⁶ The Hong Kong version of MoCA was performed in a sub-group of the parental cohort for cognitive function screening.⁷ Subjects with no dementia (i.e., screening-negative) were defined as the lower cut-off score at 16th percentile in the age and education corrected normative data.

Primary-6: The CUHK volunteer-based cohort was aimed to recruit individuals without ocular abnormalities except for mild cataract as a control group for comparison of ocular imaging measurements with glaucoma. All subjects underwent a comprehensive ophthalmic examination, and cognitive function screening was performed using the Hong Kong version of MoCA in the cohort same as Primary-5.

The inventors obtained 5 non-overlapping datasets for testing dated from 25 Jul. 2011 to 1 Jun. 2021. Included were 3 independent and retrospectively collected datasets of retinal photographs with amyloid-PET scans examination for further assessing the discriminative ability of the deep learning algorithm between groups with Aβ-positive and Aβ-negative, in addition to the label of Alzheimer's disease-dementia/no dementia. All subjects received 11C-Pittsburgh compound B (PiB) intravenously and underwent PET imaging for quantifying the amount of Aβ deposition in a series of brain regions. In addition, the inventors obtained 2 further independent and retrospectively collected datasets, one from a clinic, and another from a community-based study for further assessing the discriminative ability of the DL algorithm between groups with Alzheimer's disease-dementia and no dementia. These 5 studies were collected from:
Testing-1: Amyloid Brain and Retinal Imaging (ABRI) Study, Singapore
Testing-2: The Chinese University of Hong Kong—Screening for Early Alzheimer's DiseaSe (CU-SEEDS) study, Hong Kong
Testing-3: Mayo Clinic, Rochester, MN, USA
Testing-4: Mayo Clinic College of Medicine and Science, Scottsdale, AZ, USA
Testing-5: Mr. OS and Ms. OS Hong Kong Study, Hong Kong

Testing-1: The ABRI study is a subgroup of the Harmonization Cohort study, PET-MR imaging was performed on a mMR synchronous PET/MR scanner (Siemens Healthcare GmbH) at the Clinical Imaging Research Centre of the National University of Singapore.⁸ PET images were obtained at 40-min post-injection. Aβ-positive was defined from visual interpretation by experts from 6 Alzheimer's disease-specific regions: frontal lobe, parietal lobe, temporal lobe, anterior cingulate, praecuneus/posterior cingulate.

Testing-2: The participants of the CU-SEEDS study were recruited from the community and from the Cognitive Disorder Clinic of the Prince of Wales Hospital, Hong Kong, to validate different biomarkers (e.g., brain MRI, plasma) for detection of Alzheimer's disease.⁹ PET/CT imaging was performed at the Department of Nuclear Medicine & PET of Hong Kong Sanatorium & Hospital, Hong Kong. PET images were obtained at 35 min post-injection. Aβ-positive was defined as 1) increased ¹¹C-PIB uptake was visually observed in regions known to have amyloid-beta deposits in patients with Alzheimer's disease-dementia, e.g., frontal lobe, parietal lobe, lateral temporal lobe, posterior cingulate, praecuneus and/or caudate; and/or 2) global retention ≥1.42.

Testing-3: The participants were recruited from the Mayo Clinic Study of Aging and Alzheimer's Disease Research Centre at Mayo Clinic Rochester. In this dataset, all participants underwent PET/CT imaging. Amyloid PET imaging was performed with Pittsburgh compound B. PET images were analyzed with the institution's in-house, fully automated image-processing pipeline, where image voxel values are extracted from automatically labelled regions of interest propagated from an MRI template.¹⁰ A PiB standardized uptake value ratio for each participant was calculated as previously described.¹¹

Testing-4: The participants were recruited from the Behavioural Neurology Clinic at Mayo Clinic Arizona and National institute of Aging Arizona Alzheimer's Disease Research Core Centre.

Testing-5: The participants aged ≥65 years from the Mr. OS and Ms. OS Hong Kong Study were mainly recruited through posting advertisement in housing estates and local community centers from August 2001 to December 2003.¹² The participants were invited to follow up from 2019 to 2021 for examination including retinal photography and cognitive screening testing using the Hong Kong version of MoCA at the Jockey Club Centre for Osteoporosis Care and Control, the Chinese University of Hong Kong. Participants who were screening-positive were referred to a geriatrician for further clinical examination.¹³

Definition and labelling of Alzheimer's disease-dementia, cognitive impairment no dementia (CIND), and no dementia proceeded as follows. All the individuals were labelled as “Alzheimer's disease-dementia” and “no dementia” (or “cognitive impairment no dementia (CIND)”), based on a neuropsychological assessment or cognitive function screening test. Individuals with Alzheimer's disease-dementia in the Primary-1, Primary-2, Primary-3, Testing-1 to -5 fulfilled Diagnostic and Statistical Manual of Mental Disorders, 4th edition (DSM-IV) criteria for dementia syndrome (Alzheimer's type) and National Institute of Neurological and Communicative Disorders and Stroke and the Alzheimer's Disease and Related Disorders Association (NINDS-ADRDA) criteria for probable or possible Alzheimer's disease. In the Testing-1 to -3 datasets, CIND was defined as impairment on neuropsychological assessment but did not meet the criteria for dementia according to the DSM-IV, and subjects without dementia or had no cognitive impairment was defined as no objective impairment on the neuropsychological assessment. In Primary-4, Primary-5, Primary-6, Testing-4, and Testing-5, subjects with “no dementia” were selected and defined as AMT or MoCA screening-negative without any history of neurodegenerative diseases.

For deep learning model development, the inventors presented a whole framework for Alzheimer's Disease classification from retinal photographs by utilizing novel domain adaptation technique^{14,15}. Two key points are introduced: the fusion mechanism for multiple retinal photographs and domain adaptation mechanism for domain discrepancy, respectively.

In the provided bilateral model, the classification label used for Alzheimer's disease-dementia is subject-level while the retinal photography is taken at eye-level. Therefore, one-to-one mapping between a retinal photograph and a disease-label is difficult to be guaranteed. While not being bound by theory, the inventors hypothesized that the Alzheimer's disease-related features on retinal photographs are present on either one eye or both eyes. Based on this hypothesis, the inventors provided a novel “bilateral” deep learning model to classify Alzheimer's disease through retinal photographs from both eyes.

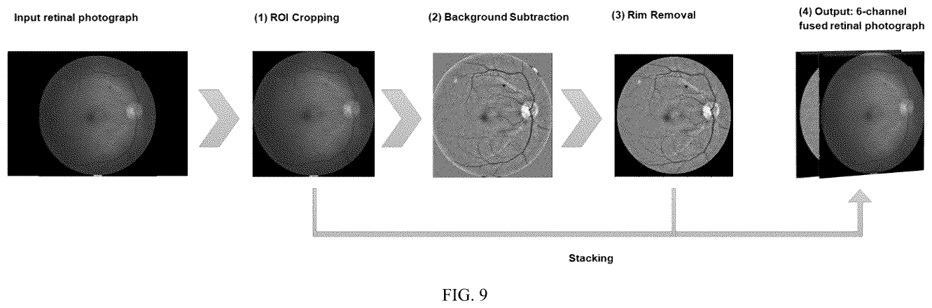

The model first applied image pre-processing methods, including data normalization and data augmentation on the retinal photographs before model development, as the retinal photographs that were collected from different centers using different retinal cameras and imaging protocols (FIG. 9). Second, the provided model used EfficientNet-b2¹⁶ as the backbone feature extractor, and then equipped the network with domain adaptation technique to deal with dataset discrepancy concerns.¹⁷ As the labelling input and classification output were subject-level, rather than image-level, the system was configured and adapted to integrate Alzheimer's disease-related features from multiple retinal photographs of right-eye and left-eye and different fields (i.e., optic nerve head-centered and macula-centered) to extract the hidden features and used fusion methods to integrate information from different feature extractors for Alzheimer's disease classification for each study subject.

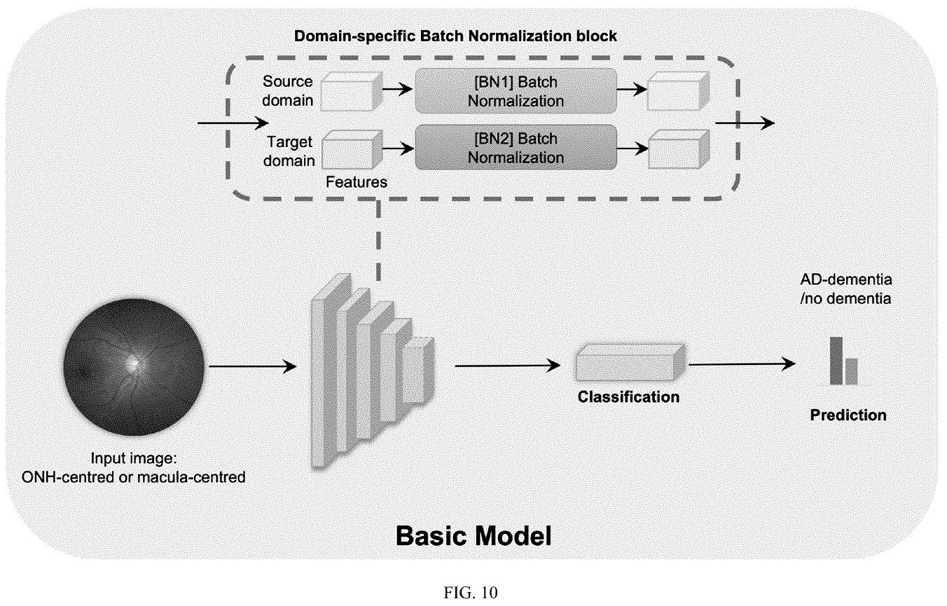

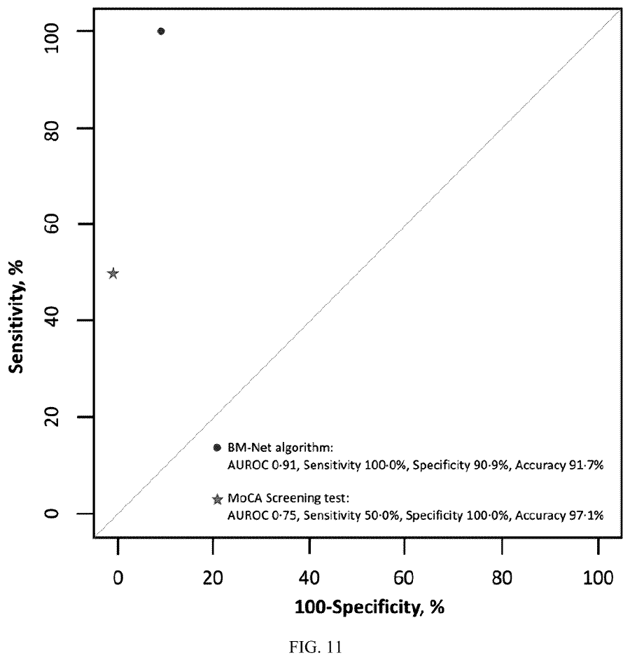

The provided method consists of three phases for usage under different conditions. In the first phase, a bilateral model (“BM-Net”) for dual eye analysis took both eyes' hidden features into account. This was first designed a basic model and only used one retinal photograph for the classification network (see FIG. 10). On top of the basic model, the BM-Net considered the joint information of optic nerve head-centered and macula-centered retinal photographs from both eyes (see FIG. 12A). Theoretically, the BM-Net made predictions with the assumption that either or both eyes have Alzheimer's disease features appeared. The inventors concatenated the features from different eyes and applied additional convolutional layers to fuse them for the final Alzheimer's disease classification.

In this embodiment, the provided bilateral model (BM-Net) takes the hidden features from retinal photographs in both eyes into account. Theoretically, the BM-Net made predictions with the assumption that either or both eyes have Alzheimer's disease-related retinal features are present. The inventors concatenate the features from different eyes and apply additional convolutional layers to fuse them for the final classification.

In this embodiment the inventors provided a unilateral model (“UM-Net”) for single eye analysis as individuals could have ungradable retinal photographs from one eye (e.g., due to severe cataract) but gradable retinal photographs from another eye (see FIG. 12B). Embodiments would also accept input of retinal photographs from one eye and output a subject-level classification (i.e., Alzheimer's disease-dementia/no dementia). Clinically, the acquisition of retinal photographs for each individual normally takes two fields of view (i.e., optic nerve head-centered and macula-centered) from each eye. To properly fuse the information from both two of fields, the inventors provided a unilateral model (UM-Net) to classify Alzheimer's disease-dementia using the fused features in each eye from both optic nerve head-centered and macula-centered retinal photographs. This model assumed the targeted unilateral eye has Alzheimer's disease related retinal features present.

Based on the basic network, the UM-net considered the joint information of optic nerve head-centered and macula-centered retinal photographs. Similar to multi-view images, the optic nerve head-centered and macula-centered retinal photographs have intersected and non-intersected areas. This embodiment adopted the Multi-view CNN^{18,19} method which used a shared feature extractor ahead of information fusion. This embodiment used 3D convolution operation for feature map aggregation in the fusion layer to take the advantage of the strict imaging protocol of retinal photographs. The framework is depicted in FIG. 12A.

To explore the added values of demographic and clinical information (age, gender, yes/no hypertension, yes/no diabetes) to Alzheimer's disease classification results, the inventors further designed a hybrid model with demographic and clinical information (HM-Net) to integrate the information and high-level semantic features extracted from the deep model layers. Specifically, the inventors applied Bilinear Transformation to realize it as illustrated in FIG. 12C. (see https://pytorch.org/docs/stable/generated/torch.nn.Bilinear.html)

The inventors finally trained a “risk factors alone” model for Alzheimer's disease prediction. The risk factors include age, gender, yes/no diabetes, and yes/no hypertension. The inventors also utilized deep learning to model the relationship between risk factors and Alzheimer's disease. The deep learning model consisted of 3 fully-connected layers with 128, 256, and 2 nodes, respectively. After the first two fully-connected layers, the inventors added dropout layer with a ratio of 0.25. The SoftMax was employed after the output to normalize the prediction between 0-1. The inventors trained this model for 1000 epochs with a batch size of 128 on one Tesla V100 GPU. The learning rate was initially set as 0.001 and decreased by 0.1 after every 400 epochs while Adam optimizer was applied.

Retinal features on retinal photographs can appear differently due to different retinal cameras, imaging protocols, ethnicity, and ocular pathologies, etc. Such dataset discrepancy leads to the poor performance of the deep learning models on new unseen datasets. In this study, the inventors defined the training datasets as “source domain” and testing datasets as “target domain” to tackle the issue of dataset discrepancy. The inventors first utilized EfficientNet-b2¹⁶ as the backbone to extract features, and then equipped with domain adaptation technique to deal with dataset discrepancy problem via a domain adaptation mechanism. Specifically, the whole framework consists of two stages. In the first stage, the inventors trained the deep models (Bilateral model or Unilateral model) with supervised learning on the source dataset with image-level annotations (Alzheimer's disease-dementia or no dementia). In the second stage, the inventors introduced a domain adaptation method by estimating the pseudo labels for the retinal photographs from the target domain using domain-specific batch normalization technique.¹⁷ According to the pseudo labels, the network can learn the domain-specific information through the multi-task learning paradigm. During training process, images from the source and target domains were fed into separate batch normalization layers (FIGS. 12A-12C). The remaining layers for feature extraction are shared between images from different domains for the domain dependent feature learning. Through the fusion of the domain independent and domain dependent knowledge learning, the deep models could transfer the discriminative features from labelled source domain to the unlabeled target domain. Therefore, the classification performance on the target domain is generally improved as shown in Table 4-4.

The inventors employed cross validation on testing datasets during the domain adaptation period. Specifically, for the internal dataset, the inventors split the data into internal training and internal validation sets with a ratio of 4:1. The inventors then divided each testing dataset into 5 folds and utilized 4 folds without labels combining with the internal training set to train the model. Thus, there were 5 different models trained with different folds. The inventors tested each model on the same internal validation set and the remaining fold of each testing dataset. The final performance was the average of the five models. The unsupervised domain adaptation process was done on the “training folds” of each testing dataset.

In terms of model training, the inventors applied a series of steps to deal with the highly unbalanced dataset and the large volume of parameters. To address the data imbalance issue, the inventors used an over-sampling strategy to select an equal number of Alzheimer's disease-dementia and control subjects for the network training at each epoch. Furthermore, for each subject, the provided model was configured to randomly select 4 images across different visits (random repeating if not enough). Thus, the inventors inhibited or avoided data leakage while also maximizing the data variance in each epoch of training.

The inventors trained the network with binary cross-entropy loss in both stages for the bilateral model and unilateral model. To deal with the class imbalance problem, the inventors utilized over sampling for the class with less data, so that there was no need to apply weighted loss functions. In the first stage, the objective function can be represented as

$${L}_{1} ( {X}_{a} ) = \frac{1}{N} {\sum }_{i = 1}^{N} - {y}_{i} * log ( {p}_{i} ( {y}_{i} = 1 \vert {θ}_{ℏ} , {x}_{i} ∈ {X}_{a} ) ) - ( 1 , {y}_{i} ) * log ( 1 - {p}_{i} ( {y}_{i} = 0 \vert {θ}_{ℏ} , {x}_{i} ∈ {X}_{a} ) ) ,$$

where N is the total number of paired retinal photographs (i.e., both optic nerve head-centered and macula-centered retinal photographs in the eye are available) in the source domain, yᵢ is the classification label of the i-th image pairs, and pᵢ is the prediction probability of current subject which is normalized by Softmax function, and θₛ represents the network parameter updated for source domain dataset. The paired retinal photographs were sampled from the same patients. The number of paired retinal photographs can be different according to the unilateral model (two images) or bilateral model (four images).

In the second stage, the inventors expected the network could learn the knowledge from both source and target domains. Therefore, the inventors estimated the pseudo labels of target domain images using the network weight trained on the first stage, so that the model can be trained with full supervision L:

$$L = {L}_{1} ( {X}_{a} ) + {L}_{2} ( {X}_{t} ) ,$$

$$and ,$$

$${L}_{2} ( {X}_{t} ) = \frac{1}{M} {\sum }_{j = 1}^{M} - {y}_{j}^{′} * log ( {p}_{j} ( {y}_{j}^{′} = 1 \vert {θ}_{t} , {x}_{j} ∈ {X}_{x} ) ) - ( 1 , {y}_{j}^{′} ) * log ( 1 - {p}_{j} ( {y}_{j}^{′} = 0 \vert {θ}_{t} , {x}_{j} ∈ {X}_{t} ) ) ,$$

where M is the total number of paired retinal photographs in the target domain, yⱼ′ is the pseudo label of j-th paired retinal photographs, and pⱼ is the prediction probability, and θₜ represents the network parameter updated for target domain dataset. It is worth to note that the L₁ is minimized to optimize the whole convolutional layers and batch normalization layer for the source domain, while L₂ is minimized to optimize the whole convolutional layers and batch normalization layer for the target domain.

Since the pseudo labels contain noisy labels, the inventors updated pseudo labels on each epoch training in the second stage according to

$${y}^{″} = ( 1 - λ ) * {y}^{′} + λ * {y}_{c} ,$$

where y′ is the pseudo label used for previous epoch, y_{c} is the new calculated pseudo label in the current epoch, and y″ is the new pseudo label for the network training in the next epoch. λ is a balance coefficient and is calculated as follows.

$$λ = 2. / ( 1. + {e}^{( - 5 * current epoch / total epoch )} ) - 1. .$$

Experimental implementation details included that the deep learning algorithm was implemented with the PyTorch library. The inventors train the bilateral model, unilateral model, and hybrid model with a batch size of 40, 60, and 40, respectively and a total of 160 epochs for domain adaptation training. The inventors utilized RAdam²⁰ as the optimizer. The data augmentation is deployed in a random way including affine, horizontal and vertical flip, and color jitter.

To aid visualization of the features used for the classification and to better understand the discriminative features among Alzheimer's disease-dementia subjects out of non-demented subjects, the inventors used Gradient-weighted Class Activation Mapping (Grad-CAM) to visualize the discriminative features used for the classification. Since the inventors developed a multi-input model, the weighted gradients were generated from the last shared convolution layer from the basic model. Moreover, to adequately demonstrate the attention areas, the inventors used eye-wise normalization, which was used to investigate the attention on different eyes.

The training included two stages. While training in the first stage, SGD Optimizer was used with 0.001 learning rate and 0.00002 weight decay, along with the binary cross-entropy loss. In the second stage, the learning rate is decreased to 0.0001 to fine-tune the network on the target domain dataset with a hybrid loss function for both source and target domains. Specifically, the inventors used Kornia for GPU-based data augmentation to improve the data augmentation speed due to the massive parallel input images. The network was trained on two GPUs of NVIDIA Tesla V100 with CUDA v10.1.

Supplementary Table 4-1. Description and characteristics of the primary
training/validation and testing datasets at visit-level. For the subjects
with follow-up visit, the duration between visits was around 12-month.

| | Number | | Number | |
|---|---|---|---|---|
| | of | | of | |
| | subjects | | *paired RP | Number of |
| | with | | in subjects | *paired RP in |
| Primary training/ | follow- | | with AD | subjects with |
| validation dataset | up visit | Visit | dementia | no dementia |
| Memory Aging and | 145 | 1 | 2,835 | 746 |
| Cognition Centre | (38.4%) | 2 | 674 | 166 |
| (Harmonization cohort), | | 3 | 417 | 76 |
| Singapore | | 4 | 216 | 25 |
| | | 5 | 46 | 0 |
| | | 6 | 18 | 0 |
| Study of Novel Retinal | 49 | 1 | 42 | 51 |
| Imaging Biomarkers for | (64.5%) | 2 | 17 | 65 |
| Cognitive Decline, | | 3 | 4 | 8 |
| Hong Kong | | 4 | 19 | 7 |
| | | 5 | 1 | 1 |
| Queen's University of | 0 (0%) | 1 | 1,057 | 2,678 |
| Belfast, United Kingdom | | | | |
| The Singapore | 0 (0%) | 1 | 0 | 2,099 |
| Epidemiology of Eye | | | | |
| Diseases study, | | | | |
| Singapore | | | | |
| Parental cohort of | 0 (0%) | 1 | 0 | 815 |
| Hong Kong | | | | |
| Children Eye Study, | | | | |
| Hong Kong | | | | |
| CUHK volunteer cohort, | 0 (0%) | 1 | 0 | 49 |
| Hong Kong | | | | |
| | | | | Number of |
| | | | Number | *paired |
| | Number | | of | retinal |
| | of | | *paired RP | photographs |
| | subjects | | in subjects | in subjects |
| | with | | with Aβ- | with |
| Testing datasets with | follow- | | positive | Aβ-negative |
| amyloid-PET imaging | up visit | Visit | on PET | on PET |
| Amyloid Brain and | 0 (0%) | 1 | 155 | 337 |
| Retinal Imaging (ABRI) | | | | |
| Study, Singapore | | | | |
| (Testing-1) | | | | |
| Screening for Early | 0 (0%) | 1 | 36 | 31 |
| Alzheimer's Disease | | | | |
| (SEEDS) Study, | | | | |
| Hong Kong (Testing-2) | | | | |
| Mayo Clinic Rochester, | 0 (0%) | 1 | 42 | 69 |
| US (Testing-3) | | | | |
| | Number | | | |
| | of | | Number of | Number of |
| | subjects | | *paired RP | *paired RP |
| Testing datasets | with | | in subjects | in subjects |
| without amyloid-PET | follow- | | with AD | with no |
| imaging | up visit | Visit | dementia | dementia |
| Mayo Clinic Arizona, | 0 (0%) | 1 | 14 | 17 |
| US (Testing-4) | | | | |
| Mayo Clinic Arizona, | 0 (0%) | 1 | 14 | 17 |
| US (Testing-4) | | | | |
| Mr. OS and Ms. OS | 1 (1.1%) | 1 | 4 | 111 |
| Hong Kong Study, | | 2 | 1 | 0 |
| Hong Kong (Testing-5) | | | | |

AD = Alzheimer's disease; RP-retinal photograph.

Supplementary Table 4-2. The subject-level performance of the deep learning based unilateral
model in the internal validation and testing datasets stratified by the laterality of the eye.

| | | | |
|---|---|---|---|
| Accuracy, % | Sensitivity, % | Specificity, % | AUROC |

Unilateral model (Right Eye)
Alzheimer's disease-dementia vs. no dementia

| | | | | |
|---|---|---|---|---|
| Internal | 81.2% ± 2.0% | 80.4% ± 4.8% | 81.8% ± 4.1% | 0.88 ± 0.01 |
| validation | | | | |
| Testing-1 | 71.9% ± 9.6% | 75.0% ± 13.7% | 79.6% ± 18.8% | 0.62 ± 0.18 |
| Testing-2 | 80.2% ± 10.1% | 78.4% ± 15.7% | 88.8% ± 13.1% | 0.63 ± 0.15 |
| Testing-3 | 90.8% ± 8.6% | 90.0% ± 22.4% | 98.0% ± 4.5% | 0.83 ± 0.21 |
| Testing-4 | 88.8% ± 11.0% | 78.3% ± 21.7% | 100.0% ± 0.0% | 0.70 ± 0.20 |
| Testing-5 | 88.8% ± 14.3% | 100.0% ± 0.0% | 87.6% ± 15.7% | 0.88 ± 0.16 |

Unilateral model (Left Eye)
Alzheimer's disease-dementia vs. no dementia

| | | | | |
|---|---|---|---|---|
| Internal | 79.6% ± 1.6% | 84.6% ± 5.0% | 77.7% ± 3.4% | 0.88 ± 0.01 |
| validation | | | | |
| Testing-1 | 73.7% ± 9.2% | 69.7% ± 17.2% | 90.9% ± 20.3% | 0.69 ± 0.13 |
| Testing-2 | 86.5% ± 11.8% | 91.7% ± 16.7% | 85.8% ± 18.9% | 0.74 ± 0.19 |
| Testing-3 | 89.2% ± 10.9% | 93.3% ± 14.9% | 91.0% ± 12.4% | 0.84 ± 0.13 |
| Testing-4 | 80.3% ± 12.3% | 90.0% ± 22.4% | 80.0% ± 20.9% | 0.61 ± 0.14 |
| Testing-5 | 69.5% ± 16.0% | 100.0% ± 0.0% | 67.3% ± 16.9% | 0.67 ± 0.17 |

Note:
Five-fold cross-validation method was applied in each testing dataset. All the metrics were reported as mean ± standard deviation.
AUROC = area under the receiver operating characteristic curve.

Supplementary Table 4-3. The paired image-level performance of the deep learning based
bilateral model, unilateral model, hybrid model and risk factor alone model in the internal
validation and testing datasets.

| | | | |
|---|---|---|---|
| Accuracy, % | Sensitivity, % | Specificity, % | AUROC |

Bilateral model
AD-dementia vs. no dementia

| | | | | |
|---|---|---|---|---|
| Internal | 80.0% ± 2.8% | 75.1% ± 2.2% | 82.6% ± 4.4% | 0.84 ± 0.02 |
| validation | | | | |
| Testing-1 | 81.3% ± 9.3% | 73.8% ± 15.1% | 95.8% ± 9.3% | 0.81 ± 0.14 |
| Testing-2 | 89.3% ± 13.7% | 91.7% ± 16.7% | 90.0% ± 20.0% | 0.80 ± 0.24 |
| Testing-3 | 83.5% ± 17.8% | 87.8% ± 21.7% | 99.7% ± 0.7% | 0.79 ± 0.20 |
| Testing-4 | 79.5% ± 22.4% | 85.0% ± 22.4% | 90.0% ± 22.4% | 0.73 ± 0.29 |
| Testing-5 | 89.6% ± 4.2% | 100.0% ± 0.0% | 88.6% ± 4.7% | 0.89 ± 0.05 |

Aβ-positive vs. Aβ-negative

| | | | | |
|---|---|---|---|---|
| Testing-1 | 81.7% ± 9.4% | 76.5% ± 17.9% | 87.6% ± 13.2% | 0.76 ± 0.16 |
| Testing-2 | 89.3% ± 13.7% | 90.0% ± 20.0% | 93.8% ± 12.5% | 0.86 ± 0.16 |
| Testing-3 | 79.7% ± 18.0% | 80.0% ± 21.6% | 100.0% ± 0.0% | 0.75 ± 0.19 |

Aβ-positive (clinically diagnosed AD-dementia cases only) vs. Aβ-negative
(no cognitive impairment controls only)

| | | | | |
|---|---|---|---|---|
| Testing-1 | 87.2% ± 14.9% | 79.5% ± 23.7% | 96.9% ± 6.3% | 0.81 ± 0.22 |
| Testing-2 | 90.8% ± 10.7% | 91.7% ± 16.7% | 93.8% ± 12.5% | 0.85 ± 0.17 |
| Testing-3 | 79.7% ± 22.6% | 72.2% ± 26.1% | 100.0% ± 0.0% | 0.68 ± 0.22 |

Unilateral model
AD-dementia vs. no dementia

| | | | | |
|---|---|---|---|---|
| Internal | 80.1% ± 2.2% | 81.2% ± 5.1% | 79.6% ± 4.4% | 0.88 ± 0.01 |
| validation | | | | |
| Testing-1 | 65.9% ± 5.9% | 58.9% ± 17.4% | 86.8% ± 19.7% | 0.64 ± 0.11 |
| Testing-2 | 80.3% ± 13.4% | 84.7% ± 13.9% | 82.5% ± 23.6% | 0.71 ± 0.14 |
| Testing-3 | 84.5% ± 9.4% | 88.3% ± 12.6% | 86.0% ± 15.7% | 0.79 ± 0.15 |
| Testing-4 | 79.9% ± 6.8% | 69.3% ± 21.3% | 92.5% ± 11.2% | 0.67 ± 0.05 |
| Testing-5 | 72.7% ± 10.3% | 100.0% ± 0.0% | 70.4% ± 10.7% | 0.76 ± 0.10 |

Aβ-positive vs. Aβ-negative

| | | | | |
|---|---|---|---|---|
| Testing-1 | 71.3% ± 12.1% | 68.6% ± 18.1% | 82.4% ± 20.5% | 0.66 ± 0.17 |
| Testing-2 | 75.0% ± 8.9% | 78.3% ± 20.8% | 74.9% ± 25.1% | 0.64 ± 0.20 |
| Testing-3 | 83.7% ± 11.7% | 80.7% ± 11.8% | 93.8% ± 12.5% | 0.80 ± 0.16 |

Aβ-positive (clinically diagnosed AD-dementia cases only) vs. Aβ-negative
(no cognitive impairment controls only)

| | | | | |
|---|---|---|---|---|
| Testing-1 | 67.2% ± 9.4% | 66.7% ± 17.9% | 80.1% ± 24.4% | 0.62 ± 0.17 |
| Testing-2 | 79.3% ± 15.2% | 84.7% ± 13.9% | 80.2% ± 29.5% | 0.70 ± 0.18 |
| Testing-3 | 88.7% ± 11.4% | 87.2% ± 11.7% | 100.0% ± 0.0% | 0.86 ± 0.16 |

Hybrid model
AD-dementia vs. no dementia

| | | | | |
|---|---|---|---|---|
| Internal | 77.0% ± 3.5% | 84.5% ± 5.6% | 73.0% ± 6.6% | 0.82 ± 0.03 |
| validation | | | | |
| Testing-1 | 79.1% ± 8.6% | 83.2% ± 10.1% | 80.3% ± 25.9% | 0.76 ± 0.17 |
| Testing-2 | 79.3% ± 7.4% | 91.8% ± 14.4% | 80.0% ± 27.4% | 0.63 ± 0.18 |
| Testing-3 | 88.9% ± 15.8% | 90.9% ± 17.4% | 99.7% ± 0.7% | 0.84 ± 0.21 |
| Testing-4 | 85.3% ± 10.7% | 80.0% ± 27.4% | 93.3% ± 14.9% | 0.72 ± 0.23 |
| Testing-5 | 96.2% ± 3.3% | 100.0% ± 0.0% | 95.9% ± 3.6% | 0.96 ± 0.04 |

Aβ-positive vs. Aβ-negative

| | | | | |
|---|---|---|---|---|
| Testing-1 | 71.5% ± 8.2% | 73.8% ± 17.5% | 70.3% ± 15.0% | 0.62 ± 0.14 |
| Testing-2 | 81.3% ± 6.8% | 84.8% ± 14.6% | 81.3% ± 23.9% | 0.67 ± 0.28 |
| Testing-3 | 87.5% ± 20.7% | 85.0% ± 21.8% | 100.0% ± 0.0% | 0.86 ± 0.22 |

Aβ-positive (clinically diagnosed AD-dementia cases only) vs. Aβ-negative
(no cognitive impairment controls only)

| | | | | |
|---|---|---|---|---|
| Testing-1 | 77.7% ± 8.2% | 78.9% ± 13.7% | 83.1% ± 24.4% | 0.72 ± 0.16 |
| Testing-2 | 80.6% ± 5.5% | 91.8% ± 14.4% | 80.0% ± 27.4% | 0.63 ± 0.17 |
| Testing-3 | 90.6% ± 18.0% | 88.6% ± 19.2% | 100.0% ± 0.0% | 0.89 ± 0.20 |

“Risk factors alone” model
AD-dementia vs. no dementia

| | | | | |
|---|---|---|---|---|
| Internal | 79.0% ± 0.8% | 78.4% ± 1.0% | 79.3% ± 1.0% | 0.81 ± 0.00 |
| validation | | | | |
| Testing-1 | 75.9% ± 8.7% | 81.6% ± 20.6% | 85.0% ± 22.4% | 0.61 ± 0.19 |
| Testing-2 | 80.2% ± 21.2% | 80.0% ± 29.8% | 86.0% ± 21.9% | 0.76 ± 0.23 |
| Testing-3 | 81.7% ± 29.1% | 83.3% ± 23.6% | 85.0% ± 33.5% | 0.76 ± 0.25 |
| Testing-4 | 67.1% ± 16.2% | 80.0% ± 27.4% | 66.7% ± 33.4% | 0.58 ± 0.08 |
| Testing-5 | 91.7% ± 8.4% | 100.0% ± 0.0% | 90.9% ± 9.1% | 0.91 ± 0.09 |

Aβ-positive vs. Aβ-negative

| | | | | |
|---|---|---|---|---|
| Testing-1 | 73.5% ± 6.1% | 85.1% ± 26.0% | 70.0% ± 20.9% | 0.59 ± 0.11 |
| Testing-2 | 80.0% ± 21.6% | 76.0% ± 27.7% | 86.0% ± 21.9% | 0.74 ± 0.22 |
| Testing-3 | 81.3% ± 14.2% | 76.7% ± 17.6% | 100.0% ± 0.0% | 0.74 ± 0.13 |

Aβ-positive (clinically diagnosed AD-dementia cases only) vs. Aβ-negative
(no cognitive impairment controls only)

| | | | | |
|---|---|---|---|---|
| Testing-1 | 80.6% ± 5.8% | 88.7% ± 17.6% | 85.0% ± 22.4% | 0.68 ± 0.17 |
| Testing-2 | 84.6% ± 17.7% | 86.7% ± 18.2% | 86.0% ± 21.9% | 0.75 ± 0.22 |
| Testing-3 | 79.2% ± 14.4% | 70.9% ± 21.0% | 100.0% ± 0.0% | 0.65 ± 0.11 |

AD-Alzheimer's disease; AUROC = area under the receiver operating characteristic curve.

Supplementary Table 4-4. The performance of the proposed bilateral model for the
classification of Alzheimer's disease-dementia/no dementia subjects, before and after
employing an unsupervised domain adaptation with domain-specific batch normalization
in all the testing datasets.

| | | | |
|---|---|---|---|
| Accuracy, % | Sensitivity, % | Specificity, % | AUROC |

Before domain adaptation

| | | | | |
|---|---|---|---|---|
| Testing-1 | 75.3% ± 10.5% | 72.9% ± 22.6% | 89.1% ± 14.0% | 0.76 ± 0.15 |
| Testing-2 | 83.3% ± 12.2% | 86.7% ± 18.2% | 82.0% ± 24.9% | 0.64 ± 0.21 |
| Testing-3 | 85.4% ± 8.4% | 94.9% ± 8.7% | 85.2% ± 13.6% | 0.78 ± 0.16 |
| Testing-4 | 81.7% ± 11.4% | 67.5% ± 20.9% | 100.0% ± 0.0% | 0.68 ± 0.21 |
| Testing-5 | 97.6% ± 4.1% | 100.0% ± 0.0% | 97.4% ± 4.4% | 0.97 ± 0.04 |

After domain adaptation

| | | | | |
|---|---|---|---|---|
| Testing-1 | 81.3% ± 9.3% | 73.8% ± 15.1% | 95.8% ± 9.3% | 0.81 ± 0.14 |
| Testing-2 | 89.3% ± 13.7% | 91.7% ± 16.7% | 90.0% ± 20.0% | 0.80 ± 0.24 |
| Testing-3 | 83.5% ± 17.8% | 87.8% ± 21.7% | 99.7% ± 0.7% | 0.79 ± 0.20 |
| Testing-4 | 79.5% ± 22.4% | 85.0% ± 22.4% | 90.0% ± 22.4% | 0.73 ± 0.29 |
| Testing-5 | 89.6% ± 4.2% | 100.0% ± 0.0% | 88.6% ± 4.7% | 0.89 ± 0.05 |

Note:
Five-fold cross-validation method was applied in each testing dataset. All the metrics were reported as mean ± standard deviation.
AUROC = area under the receiver operating characteristic curve.

Supplementary Table 4-5. The cross tabulation of the bilateral and
unilateral models for the classification of AD-dementia/no dementia
at both subject-level and image-level in the internal validation.

| | | | | |
|---|---|---|---|---|
| Ture | True | False | False | |
| positive | negative | negative | positive | Total |

Bilateral model
Subject-level

| | | | | | |
|---|---|---|---|---|---|
| Folder 1 | 68 | 389 | 6 | 72 | 535 |
| Folder 2 | 70 | 376 | 4 | 85 | 535 |
| Folder 3 | 69 | 387 | 5 | 74 | 535 |
| Folder 4 | 71 | 352 | 3 | 109 | 535 |
| Folder 5 | 68 | 386 | 6 | 75 | 535 |

Image-level

| | | | | | |
|---|---|---|---|---|---|
| Folder 1 | 595 | 1188 | 178 | 270 | 2231 |
| Folder 2 | 574 | 1243 | 199 | 215 | 2231 |
| Folder 3 | 575 | 1104 | 198 | 354 | 2231 |
| Folder 4 | 560 | 1261 | 213 | 197 | 2231 |
| Folder 5 | 592 | 1232 | 181 | 226 | 2231 |

Unilateral model
Subject-level

| | | | | | |
|---|---|---|---|---|---|
| Folder 1 | 146 | 919 | 24 | 145 | 1234 |
| Folder 2 | 163 | 835 | 7 | 229 | 1234 |
| Folder 3 | 157 | 861 | 13 | 203 | 1234 |
| Folder 4 | 148 | 880 | 22 | 184 | 1234 |
| Folder 5 | 150 | 923 | 20 | 141 | 1234 |

Image-level

| | | | | | |
|---|---|---|---|---|---|
| Folder 1 | 441 | 1253 | 113 | 306 | 2113 |
| Folder 2 | 413 | 1343 | 141 | 216 | 2113 |
| Folder 3 | 466 | 1250 | 88 | 309 | 2113 |
| Folder 4 | 447 | 1189 | 107 | 370 | 2113 |
| Folder 5 | 487 | 1171 | 67 | 388 | 2113 |

Note:
The inventors only employed cross validation on testing datasets during the domain adaptation period. Specifically, for the internal dataset, the inventors split the data into internal training and internal validation sets with a ratio of 4:1. The inventors then divided each external dataset into 5 folds and utilized 4 folds without labels combining with the internal training set to train the model. Thus, there were 5 different models trained with different folds. The inventors tested each model on the same internal validation set.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES FOR EXAMPLE 4: SUPPLEMENTARY APPENDIX

1. Gyanwali B, Shaik M A, Venketasubramanian N, Chen C, Hilal S. Mixed-Location Cerebral Microbleeds: An Imaging Biomarker for Cerebrovascular Pathology in Cognitive Impairment and Dementia in a Memory Clinic Population. J Alzheimers Dis 2019; 71(4): 1309-20.
2. van Veluw S J, Hilal S, Kuijf H J, et al. Cortical microinfarcts on 3T MRI: Clinical correlates in memory-clinic patients. Alzheimers Dement 2015; 11(12): 1500-9.
3. Williams M A, Silvestri V, Craig D, Passmore A P, Silvestri G. The prevalence of age-related macular degeneration in Alzheimer's disease. J Alzheimers Dis 2014; 42(3): 909-14.
4. Lim Z W, Chee M L, Soh Z D, et al. Association Between Visual Impairment and Decline in Cognitive Function in a Multiethnic Asian Population. JAMA Netw Open 2020; 3(4): be203560.
5. Fenwick E K, Gan A T L, Man R E K, et al. Vision, vision-specific functioning and mobility, and their relationship with clinically assessed cognitive impairment. Age Ageing 2021.
6. Yam J C, Tang S M, Kam K W, et al. High prevalence of myopia in children and their parents in Hong Kong Chinese Population: the Hong Kong Children Eye Study. Acta Ophthalmol 2020.
7. Wong A, Law L S, Liu W, et al. Montreal Cognitive Assessment: One Cutoff Never Fits All. Stroke 2015; 46(12): 3547-50.
8. Saridin F N, Hilal S, Villaraza S G, et al. Brain amyloid beta, cerebral small vessel disease, and cognition: A memory clinic study. Neurology 2020; 95 (21): e2845-e53.
9. Liu W, Au L W C, Abrigo J, et al. MRI-based Alzheimer's disease-resemblance atrophy index in the detection of preclinical and prodromal Alzheimer's disease. Aging (Albany NY) 2021; 13(10): 13496-514.
10. Graff-Radford J, Lesnick T, Rabinstein A A, et al. Cerebral microbleed incidence, relationship to amyloid burden: The Mayo Clinic Study of Aging. Neurology 2020; 94(2): e190-e9.
11. Jack C R, Jr., Wiste H J, Weigand S D, et al. Defining imaging biomarker cut points for brain aging and Alzheimer's disease. Alzheimers Dement 2017; 13(3): 205-16.
12. Kwok A W, Gong J S, Wang Y X, et al. Prevalence and risk factors of radiographic vertebral fractures in elderly Chinese men and women: results of Mr. OS (Hong Kong) and Ms. OS (Hong Kong) studies. Osteoporos Int 2013; 24(3): 877-85.
13. Yeung P Y, Wong L L, Chan C C, Leung J L, Yung C Y. A validation study of the Hong Kong version of Montreal Cognitive Assessment (HK-MoCA) in Chinese older adults in Hong Kong. Hong Kong Med J 2014; 20(6): 504-10.
14. Xie Q, Luong M-T, Hovy E, Le Q V. Self-training with noisy student improves imagenet classification. Proceedings of the IEEE/CVF conference on computer vision and pattern recognition; 2020; 2020. p. 10687-98.
15. Zou Y, Yu Z, Liu X, Kumar B, Wang J. Confidence regularized self-training. Proceedings of the IEEE/CVF International Conference on Computer Vision; 2019; 2019. p. 5982-91.
16. Tan M, and Le, Q. EfficientNet: Rethinking Model Scaling for Convolutional Neural Networks. Proceedings of the 36th International Conference on Machine Learning 2019.
17. Chang W G, You T, Seo S, Kwak S, Han B. Domain-Specific Batch Normalization for Unsupervised Domain Adaptation. 2019 Ieee/Cvf Conference on Computer Vision and Pattern Recognition (Cvpr 2019) 2019: 7346-54.
18. Ruder S. An overview of gradient descent optimization algorithms. ArXiv preprint arXiv: 160904747v2 2017.
19. Zhou T, Liu M, Thung K H, Shen D. Latent Representation Learning for Alzheimer's Disease Diagnosis With Incomplete Multi-Modality Neuroimaging and Genetic Data. IEEE Trans Med Imaging 2019; 38(10): 2411-22.
20. Liu L Y, Jiang H M, He P C, et al. On the Variance of the Adaptive Learning Rate and Beyond. arXiv 190803265v4 2021.
21. Selvaraju R R, Cogswell M, Das A, Vedantam R, Parikh D, Batra D. Grad-CAM: Visual Explanations from Deep Networks via Gradient-based Localization. Ieee I Conf Comp Vis 2017: 618-26.
22. Riba E, Mishkin D, Ponsa D, Rublee E, Bradski G. Kornia: an Open Source Differentiable Computer Vision Library for PyTorch. Ieee Wint Conf Appl 2020: 3663-72.

### REFERENCES

1. 2021 Alzheimer's disease facts and figures. Alzheimers Dement 2021; 17(3): 327-406.
2. Olsson B, Lautner R, Andreasson U, et al. CSF and blood biomarkers for the diagnosis of Alzheimer's disease: a systematic review and meta-analysis. Lancet Neurol 2016; 15(7): 673-84.
3. Palmqvist S, Janelidze S, Quiroz Y T, et al. Discriminative Accuracy of Plasma Phospho-tau217 for Alzheimer Disease vs Other Neurodegenerative Disorders. JAMA 2020; 324(8): 772-81.
4. Sevigny J, Chiao P, Bussiere T, et al. The antibody aducanumab reduces Abeta plaques in Alzheimer's disease. Nature 2016; 537(7618): 50-6.
5. Rabinovici G D. Controversy and Progress in Alzheimer's Disease—FDA Approval of Aducanumab. N Engl J Med 2021; 385(9): 771-4.
6. Alexander G C, Emerson S, Kesselheim A S. Evaluation of Aducanumab for Alzheimer Disease: Scientific Evidence and Regulatory Review Involving Efficacy, Safety, and Futility. JAMA 2021; 325(17): 1717-8.
7. London A, Benhar I, Schwartz M. The retina as a window to the brain-from eye research to CNS disorders. Nat Rev Neurol 2013; 9(1): 44-53.
8. La Morgia C, Ross-Cisneros F N, Koronyo Y, et al. Melanopsin retinal ganglion cell loss in Alzheimer disease. Ann Neurol 2016; 79(1): 90-109.
9. Koronyo Y, Biggs D, Barron E, et al. Retinal amyloid pathology and proof-of-concept imaging trial in Alzheimer's disease. JCI Insight 2017; 2(16).
10. Cheung C Y L, Ikram M K, Chen C, Wong T Y. Imaging retina to study dementia and stroke. Progress in Retinal and Eye Research 2017; 57:89-107.
11. Snyder P J, Alber J, Alt C, et al. Retinal imaging in Alzheimer's and neurodegenerative diseases. Alzheimers Dement 2021; 17(1): 103-11.
12. Cheung C Y, Mok V, Foster P J, Trucco E, Chen C, Wong T Y. Retinal imaging in Alzheimer's disease. J Neurol Neurosurg Psychiatry 2021; 92(9): 983-94.
13. Cheung C Y, Ong Y T, Ikram M K, et al. Microvascular network alterations in the retina of patients with Alzheimer's disease. Alzheimers Dement 2014; 10(2): 135-42.
14. Lee C S, Larson E B, Gibbons L E, et al. Associations between recent and established ophthalmic conditions and risk of Alzheimer's disease. Alzheimers Dement 2019; 15(1): 34-41.
15. LeCun Y, Bengio Y, Hinton G. Deep learning. Nature 2015; 521(7553): 436-44.
16. Ting D S W, Cheung C Y, Lim G, et al. Development and Validation of a Deep Learning System for Diabetic Retinopathy and Related Eye Diseases Using Retinal Images From Multiethnic Populations With Diabetes. JAMA 2017; 318(22): 2211-23.
17. Gulshan V, Peng L, Coram M, et al. Development and Validation of a Deep Learning Algorithm for Detection of Diabetic Retinopathy in Retinal Fundus Photographs. JAMA 2016; 316(22): 2402-10.
18. Milea D, Najjar R P, Zhubo J, et al. Artificial Intelligence to Detect Papilledema from Ocular Fundus Photographs. N Engl J Med 2020; 382(18): 1687-95.
19. Liu H, Li L, Wormstone I M, et al. Development and Validation of a Deep Learning System to Detect Glaucomatous Optic Neuropathy Using Fundus Photographs. JAMA Ophthalmol 2019; 137(12): 1353-60.
20. Burlina P M, Joshi N, Pekala M, Pacheco K D, Freund D E, Bressler N M. Automated Grading of Age-Related Macular Degeneration From Color Fundus Images Using Deep Convolutional Neural Networks. Jama Ophthalmology 2017; 135(11): 1170-6.
21. Rim T H, Lee G, Kim Y, et al. Prediction of systemic biomarkers from retinal photographs: development and validation of deep-learning algorithms. Lancet Digit Health 2020; 2(10): E526-E36.
22. Cheung C Y, Xu D, Cheng C Y, et al. A deep-learning system for the assessment of cardiovascular disease risk via the measurement of retinal-vessel calibre. Nat Biomed Eng 2021; 5(6): 498-508.
23. Rim T H, Lee C J, Tham Y C, et al. Deep-learning-based cardiovascular risk stratification using coronary artery calcium scores predicted from retinal photographs. Lancet Digit Health 2021; 3(5): E306-E16.
24. Zhang K, Liu X, Xu J, et al. Deep-learning models for the detection and incidence prediction of chronic kidney disease and type 2 diabetes from retinal fundus images. Nat Biomed Eng 2021; 5(6): 533-45.
25. Sabanayagam C, Xu D, Ting D S W, et al. A deep learning algorithm to detect chronic kidney disease from retinal photographs in community-based populations. Lancet Digit Health 2020; 2(6): e295-e302.
26. Xiao W, Huang X, Wang J H, et al. Screening and identifying hepatobiliary diseases through deep learning using ocular images: a prospective, multicentre study. Lancet Digit Health 2021; 3(2): e88-e97.
27. Abràmoff M D, Lavin P T, Birch M, Shah N, Folk J C. Pivotal trial of an autonomous AI-based diagnostic system for detection of diabetic retinopathy in primary care offices. npj Digital Medicine 2018; 1(1): 39.
28. Wang F, Casalino L P, Khullar D. Deep Learning in Medicine—Promise, Progress, and Challenges. JAMA Internal Medicine 2019; 179(3): 293-4.
29. Maddox T M, Rumsfeld J S, Payne P R O. Questions for Artificial Intelligence in Health Care. JAMA 2019; 321(1): 31-2.
30. Gunasekeran D V, Wong T Y. Artificial Intelligence in Ophthalmology in 2020: A Technology on the Cusp for Translation and Implementation. The Asia-Pacific Journal of Ophthalmology 2020; 9(2): 61-6.
31. Sharafi S M, Sylvestre J P, Chevrefils C, et al. Vascular retinal biomarkers improves the detection of the likely cerebral amyloid status from hyperspectral retinal images. Alzheimers Dement (N Y) 2019; 5:610-7.
32. Dodge S F, Karam L J. Understanding how image quality affects deep neural networks. 2016 Eighth International Conference on Quality of Multimedia Experience (QoMEX) 2016: 1-6.
33. Yip M Y T, Lim G, Lim Z W, et al. Technical and imaging factors influencing performance of deep learning systems for diabetic retinopathy. npj Digital Medicine 2020; 3(1): 40.
34. Beede E, Baylor E, Hersch F, et al. A Human-Centered Evaluation of a Deep Learning System Deployed in Clinics for the Detection of Diabetic Retinopathy. Proceedings of the 2020 CHI Conference on Human Factors in Computing Systems. Honolulu, HI, USA: Association for Computing Machinery; 2020. p. 1-12.
35. Phene S, Dunn R C, Hammel N, et al. Deep Learning and Glaucoma Specialists: The Relative Importance of Optic Disc Features to Predict Glaucoma Referral in Fundus Photographs. Ophthalmology 2019; 126(12): 1627-39.
36. Shah P, Mishra D K, Shanmugam M P, Doshi B, Jayaraj H, Ramanjulu R. Validation of Deep Convolutional Neural Network-based algorithm for detection of diabetic retinopathy—Artificial intelligence versus clinician for screening. Indian J Ophthalmol 2020; 68(2): 398-405.
37. Li F, Liu Z, Chen H, Jiang M, Zhang X, Wu Z. Automatic Detection of Diabetic Retinopathy in Retinal Fundus Photographs Based on Deep Learning Algorithm. Translational Vision Science & Technology 2019; 8(6): 4-.
38. Asaoka R, Tanito M, Shibata N, et al. Validation of a Deep Learning Model to Screen for Glaucoma Using Images from Different Fundus Cameras and Data Augmentation. Ophthalmology Glaucoma 2019; 2(4): 224-31.
39. Chalakkal R J, Abdulla W H, Thulaseedharan S S. Quality and content analysis of fundus images using deep learning. Comput Biol Med 2019; 108: 317-31.
40. Karnowski T P, Aykac D, Giancardo L, et al. Automatic detection of retina disease: robustness to image quality and localization of anatomy structure. Conf Proc IEEE Eng Med Biol Soc 2011; 2011: 5959-64.
41. Patton N, Aslam T M, MacGillivray T, et al. Retinal image analysis: concepts, applications and potential. Prog Retin Eye Res 2006; 25(1): 99-127.
42. Dai L, Wu L, Li H, et al. A deep learning system for detecting diabetic retinopathy across the disease spectrum. Nat Commun 2021; 12(1): 3242.
43. Milea D, Najjar R P, Jiang Z, et al. Artificial Intelligence to Detect Papilledema from Ocular Fundus Photographs. New England Journal of Medicine 2020; 382(18): 1687-95.
44. Li Z, He Y, Keel S, Meng W, Chang R T, He M. Efficacy of a Deep Learning System for Detecting Glaucomatous Optic Neuropathy Based on Color Fundus Photographs. Ophthalmology 2018; 125(8): 1199-206.
45. Liu H, Li L, Wormstone I M, et al. Development and Validation of a Deep Learning System to Detect Glaucomatous Optic Neuropathy Using Fundus Photographs. JAMA Ophthalmol 2019; 137(12): 1353-60.
46. Tan M, and Le, Q. EfficientNet: Rethinking Model Scaling for Convolutional Neural Networks. Proceedings of the 36th International Conference on Machine Learning 2019.
47. Selvaraju R R, Cogswell M, Das A, Vedantam R, Parikh D, Batra D. Grad-CAM: Visual Explanations from Deep Networks via Gradient-based Localization. Ieee I Conf Comp Vis 2017: 618-26.
48. Tan M, Le Q V. EfficientNet: Rethinking Model Scaling for Convolutional Neural Networks. ArXiv 2019; abs/1905.11946.
49. Sandler M, Howard A, Zhu M, Zhmoginov A, Chen L. MobileNetV2: Inverted Residuals and Linear Bottlenecks. 2018 IEEE/CVF Conference on Computer Vision and Pattern Recognition; 2018 18-23 Jun. 2018; 2018. p. 4510-20.
50. Furiak N M, Kahle-Wrobleski K, Callahan C, Klein T M, Klein R W, Siemers E R. Screening and treatment for Alzheimer's disease: predicting population-level outcomes. Alzheimers Dement 2012; 8(1): 31-8.

We claim:

1. An artificial intelligence (AI)-aided classification system for Alzheimer's disease (AD) screening from a source dataset comprising retinal photographs, the system comprising a deep learning (DL) model created by a process comprising:
a) application of one or more data pre-processing methods and one or more on-the-fly data augmentation methods to normalize the retinal photographs including region of interest (ROI) cropping,
b) background subtraction with median filters, and
c) removal of an illuminated rim and a stacking rim to create a multitude of cropped images to generate a 6-channel input for training the DL model.

2. The system according to claim 1, the DL model comprising a bilateral model, a hybrid model, and a unilateral model.

3. The system according to claim 2, wherein:
the bilateral model is trained primarily on both eyes' four images;
the hybrid model is trained primarily on both eyes' four images combing demographic information; and
the unilateral model is trained primarily on a single eye's two images.

4. The system according to claim 1, the source dataset comprising retinal photographs and demographic information derived from different centers, and the DL model comprising a feature fusion module to integrate features captured from the different centers.

5. The system according to claim 1, the DL model comprising demographic information integrated with both eyes' four images from one subject; and demographic information integrated by bilinear transformation.

6. The system according to claim 1, the DL model comprising EfficientNet-b2 as a backbone to extract features.

7. The system according to claim 6, the DL model comprising a domain adaptation technique to deal with dataset discrepancies.

8. The system according to claim 1, the DL model created by a process comprising two stages.

9. The system according to claim 8, the two stages comprising:
a first stage, trained with supervised learning on the source dataset with image-level annotations; and
a second stage, introducing a domain adaptation method by estimating the pseudo labels for the retinal photographs from the source dataset using a domain-specific batch normalization technique.

10. The system according to claim 9, wherein images from the source and target domains were fed into separate batch normalization layers in each of the first stage and the second stage, respectively.

11. The system according to claim 10, comprising an imbalance of data between a first class with more data and a second class with less data, and comprising an over sampling for the second class.

12. The system according to claim 11, comprising a training objective function utilizing both source dataset and target domain images.

13. The system according to claim 12, comprising generation of heatmaps to show the significant locations which are related to the AD with a Gradient-weighted Class Activation method.

14. The system according to claim 13, comprising pre-diagnosis image assessment and AD binary classification by a cloud-based web application.

15. A method for creating an artificial intelligence (AI)-aided classification system comprising a deep learning (DL) model for Alzheimer's disease (AD) screening from a source domain comprising retinal photographs, the method comprising:
a) applying one or more data pre-processing methods and one or more on-the-fly data augmentation methods to normalize the retinal photographs, including region of interest (ROI) cropping,
b) subtracting background with median filters, and
c) removing an illuminated rim and a stacking rim to create a multitude of cropped images to generate a 6-channel input for training the DL model;
d) training a bilateral model primarily on both eyes four images;
e) training a hybrid model primarily on both eyes' four images combing demographic information;
f) training a unilateral model primarily on a single eye's two images; and
g) applying a feature fusion module to integrate features captured from different centers; and
h) mapping from the source domain to create a target domain.

16. The method according to claim 15, comprising:
a) integrating demographic information with both eyes' four images from one subject; and
b) integrating demographic information by bilinear transformation.

17. The method according to claim 16, comprising:
i) extracting features from the source domain using EfficientNet-b2 as a backbone;
j) applying a domain adaptation technique to deal with dataset discrepancies;
k) conducting a first stage, trained with supervised learning on the source dataset with image-level annotations wherein images from the source domain and from the target domain were fed into separate batch normalization layers;
l) conducting a second stage, introducing a domain adaptation method by estimating pseudo labels for the retinal photographs from the target domain using domain-specific batch normalization technique wherein images from the source domain and from the target domain, respectively, are fed into separate batch normalization layers;
m) identifying an imbalance of data between a first class with more data and a second class with less data;
n) applying an over sampling for the second class;
o) applying a training objective function utilizing both source domain images and target domain images;
p) generating heatmaps to show the significant locations which are related to the AD with a Gradient-weighted Class Activation method; and
q) providing both a pre-diagnosis image assessment and an AD binary classification by a cloud-based web application.

18. An artificial intelligence (AI)-aided classification system for Alzheimer's disease (AD) screening from a source dataset comprising retinal photographs, the system comprising a deep learning (DL) model, the system created by a process comprising:
a) applying one or more data pre-processing methods and one or more on-the-fly data augmentation methods to normalize the retinal photographs including region of interest (ROI) cropping,
b) subtracting background with median filters, and
c) removing an illuminated rim and a stacking rim to create a multitude of cropped images to generate a 6-channel input for training the DL model;
d) training a bilateral model primarily on both eyes' four images;
e) training a hybrid model primarily on both eyes' four images combining demographic information;
f) training a unilateral model primarily on a single eye's two images;
g) applying a feature fusion module to integrate the features captured from different centers;
h) training the DL model on demographic information integrated with both eyes' four images from one subject;
i) training the DL model on demographic information integrated by bilinear transformation;
j) extracting features using EfficientNet-b2 as a backbone; and
k) applying a domain adaptation technique to resolve dataset discrepancies.

19. The system according to claim 18, wherein the DL model is created by a process comprising two stages, the two stages comprising:
conducting a first stage, trained with supervised learning on the source dataset with image-level annotations to create a target domain comprising the retinal images; and
conducting a second stage, introducing a domain adaptation method by estimating pseudo labels for the retinal photographs from the target domain using domain-specific batch normalization technique;
wherein images from the source dataset and from the target domain were fed into separate batch normalization layers in each of the first stage and the second stage, respectively; and
wherein an imbalance of data between a first class with more data and a second class with less data was balanced with over sampling for the second class.

20. The system according to claim 19, the system created by a process comprising:
l) applying a training objective function utilizing both source dataset and target domain images;
m) generating heatmaps to show the significant locations which are related to the AD with a Gradient-weighted Class Activation method; and
n) providing both pre-diagnosis image assessment and AD binary classification by a cloud-based web application.
